(19) United States
(12) Patent Application Publication
(10) Pub. No.: US 2023/0192748 A1
(43) Pub. Date: Jun. 22, 2023

(54) Etoposide Glycosides, Methods Of Making, And Uses Thereof As An Anti-Cancer Drug

(71) Applicant: DoubleRainbow Biosciences Inc., Lexington, MA (US)

(72) Inventors: Sheng Ding, Bethany, CT (US); Yasmin-Pei Chau, Cambridge, MA (US); Jacob Donald Stanley Wirth, Waltham, MA (US); Tian Xu, Guilford, CT (US); Jing-Ke Weng, Belmont, MA (US)

(21) Appl. No.: 17/906,373

(22) PCT Filed: Mar. 15, 2021
(86) PCT No.: PCT/US2021/022414

§ 371 Date: Sep. 15, 2022

Related U.S. Application Data
(60) Provisional application No. 62/990,124, filed on Mar. 16, 2020.

(51) Int. Cl. C07H 17/04

(52) U.S. Cl. CPC C07H 17/04

(57) ABSTRACT

Etoposide glycosides and methods of making etoposide glycosides are disclosed. Glycosyl transferases catalyze addition of one or more monosaccharides to etoposide to yield etoposide glycosides. Suitable monosaccharides can be in the L- or D-configuration and typically have 5, 6, or 7 carbons. Suitable monosaccharides include allose, apiose, arabinose, fructose, fucitol, fucose, galactose, glucose, glucuronic acid, mannose, A-acetylglucosamine, rhamnose, or xylose. Uridine diphosphate glycosyl transferases can catalyze formation of either an alpha or beta glycosidic bond.

### RELATED APPLICATION

This application claims the benefit of U.S. Provisional Application No. 62/990,124, filed on Mar. 16, 2020. The entire teachings of the above application are incorporated herein by reference.

### INCORPORATION BY REFERENCE OF MATERIAL IN ASCII TEXT FILE

This application incorporates by reference the Sequence Listing contained in the following ASCII text file being submitted concurrently herewith:

a) File name: 57671002001SequenceListing.txt; created Mar. 15, 2021, 18 KB in size.

### BACKGROUND

Cancer is a group of diseases characterized by uncontrolled growth and proliferation of abnormal cells that arises due a combination of genetic and environmental factors. It is the second-leading cause of death worldwide, with cancer causing 1 in 6 deaths each day (American Cancer Society 2018). With the average age of the world population on the rise, the number of new cancer cases is expected to increase.

### SUMMARY

Described herein are etoposide derivatives containing specific monosaccharide(s) or oligosaccharides(s) and methods of making these molecules utilizing enzyme catalysis. Compared to etoposide, the etoposide glycosides exhibit increased water solubility, which may contribute to improved pharmacokinetic and/or pharmacodynamic profiles. The compounds may act as prodrugs of etoposide. The compounds may exhibit improvements in potency towards inhibiting the activity of the DNA topoisomerase II protein. The compounds may exhibit enhanced therapeutic effects as an anti-cancer agent.

Thus, the present invention provides compounds that may act as prodrugs of etoposide with potential improvements in potency towards inhibiting the activity of the DNA topoisomerase II protein and enhanced anti-cancer effects.

Described herein are compounds represented by the following structural formula:

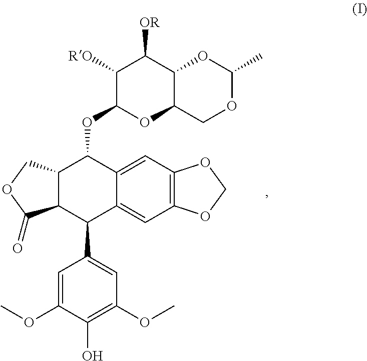

or a pharmaceutically acceptable salt thereof, wherein R and/or R′ is a monosaccharide, a disaccharide, a trisaccharide, or an oligosaccharide having 4 to 10 monosaccharides.

Described herein are pharmaceutical compositions that include an etoposide glycoside, or a pharmaceutically acceptable salt thereof, and a pharmaceutically acceptable carrier or adjuvant.

Described herein are methods of making an etoposide glycoside. The methods include: a) providing a reaction mixture; and b) allowing the reaction mixture to convert etoposide to a monosaccharide, a disaccharide, or an oligosaccharide of etoposide. The reaction mixture can include a compound having the following structural formula:

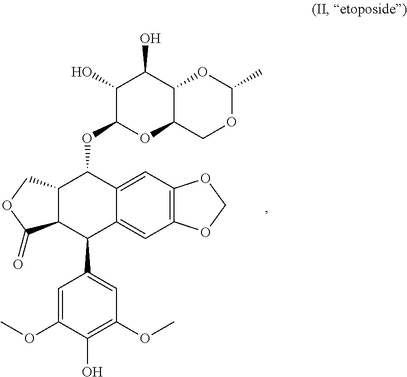

a uridine diphosphate glycosyltransferase (UGT); and uridine diphosphate-monosaccharide. The compound that is formed can have the following structural formula:

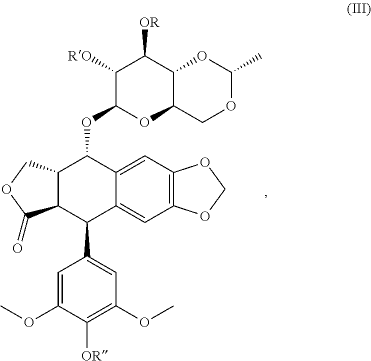

wherein R, R′, and/or R″ is a monosaccharide, a disaccharide, a trisaccharide, or an oligosaccharide having 4 to 10 monosaccharides.

In some embodiments, R, R′, and/or R″ is a monosaccharide. In some embodiments, the monosaccharide is a pentose monosaccharide, hexose monosaccharide, or heptose monosaccharide.

In some embodiments, R, R′, and/or R″ is allose, apiose, arabinose, fructose, fucitol, fucose, galactose, glucose, glucuronic acid, mannose, N-acetylglucosamine, N-acetylgalactosamine, rhamnose, or xylose. In some embodiments, R is glucosamine, galactosamine, mannosamine, 5-thio-D-glucose, nojirimycin, deoxynojirimycin, 1,5-anhydro-D-sorbitol, 2,5-anhydro-D-mannitol, 2-deoxy-D-galactose, 2-deoxy-D-glucose, 3-deoxy-D-glucose, arabinitol, galactitol, glucitol, iditol, lyxose, mannitol, L-rhamnitol, 2-deoxy-D-ribose, ribose, ribitol, ribulose, xylulose, altrose, gulose, idose, levulose, psicose, sorbose, tagatose, talose, galactal, glucal, fucal, rhamnal, arabinal, xylal, 3,4-di-O-acetyl-L-fucal, 3,4-di-O-acetyl-L-rhamnal, 3,4-di-O-acetyl-D-arabinal, 3,4-di-O-acetyl-D-xylal, valienamine, validamine, valiolamine, valienol, valienone, galacturonic acid, mannuronic acid, N-acetylneuraminic acid, N-acetylmuramic acid, gluconic acid D-lactone, galactonic acid gamma-lactone, galactonic acid delta-lactone, mannonic acid gamma-lactone, D-altro-heptulose, D-manno-heptulose, D-glycero-D-manno-heptose, D-glycero-D-gluco-heptose, D-allo-heptulose, D-altro-3-heptulose, D-glycero-D-manno-heptitol, or D-glycero-D-altro-heptitol.

In some embodiments, R, R′, and/or R″ is a disaccharide. In some embodiments, R, R′, and/or R″is a disaccharide of two glucose molecules. In some embodiments, R, R′, and/or R″ is a disaccharide of two galactose molecules. In some embodiments, R, R′, and/or R″ is a disaccharide of two xylose molecules. For any of the foregoing disaccharides, the disaccharide molecules can be bonded by a 1→2 glycosidic bond, a 1→3 glycosidic bond, or a 1→4 glycosidic bond.

In some embodiments, R, R′, and/or R″ is a trisaccharide. In some embodiments, R, R′, and/or R″ is a trisaccharide of three glucose molecules. In some embodiments, R, R′, and/or R″ is a trisaccharide of three galactose molecules. In some embodiments, R, R′, and/or R″ is a trisaccharide of three xylose molecules. For any of the foregoing trisaccharides, the trisaccharide molecules can be bonded by a 1→2 glycosidic bond and by a 1→4 glycosidic bond.

In some embodiments, the UGT includes an amino acid sequence that is at least 95% similar to SEQ ID NO: 1. In some embodiments, the UGT includes an amino acid sequence that is at least 80% similar to a region from A340 to Q382 of SEQ ID NO: 1. In some embodiments, the UGT includes an amino acid sequence that is: at least 90% similar to a region from I84 to S99 of SEQ ID NO: 1; at least 90% similar to a region from D126 to F134 of SEQ ID NO: 1; at least 90% similar to a region from L147 to S149 of SEQ ID NO: 1; and at least 80% similar to a region from A340 to Q382 of SEQ ID NO: 1.

In some embodiments, the UGT includes an amino acid sequence that is at least 95% similar to SEQ ID NO: 2. In some embodiments, the UGT includes an amino acid sequence that is at least 80% similar to a region from V278 to Q318 of SEQ ID NO: 2. In some embodiments, the UGT includes an amino acid sequence that is: at least 90% similar to a region from I67 to D75 of SEQ ID NO: 2; at least 90% similar to a region from D106 to L114 of SEQ ID NO: 2; at least 90% similar to a region from C127 to S129 of SEQ ID NO: 2; and at least 80% similar to a region from V278 to Q318 of SEQ ID NO: 2.

In some embodiments, the UGT includes an amino acid sequence that is at least 95% similar to SEQ ID NO: 3. In some embodiments, the UGT includes an amino acid sequence that is at least 80% similar to a region from V291 to Q331 of SEQ ID NO: 3. In some embodiments, the UGT includes an amino acid sequence that is: at least 90% similar to a region from W74 to V82 of SEQ ID NO: 3; at least 90% similar to a region from D111 to V119 of SEQ ID NO: 3; at least 90% similar to a region from F132 to N134 of SEQ ID NO: 3; and at least 80% similar to a region from V291 to Q331 of SEQ ID NO: 3.

In some embodiments, the UGT includes an amino acid sequence that is at least 95% similar to SEQ ID NO: 4. In some embodiments, the UGT includes an amino acid sequence that is at least 80% identical to a region from V280 to Q320 of SEQ ID NO: 4. In some embodiments, the UGT includes an amino acid sequence that is: at least 90% similar to a region from I67 to D75 of SEQ ID NO: 4; at least 90% similar to a region from D106 to L114 of SEQ ID NO: 4; at least 90% similar to a region from C127 to S129 of SEQ ID NO: 4; and at least 80% similar to a region from V280 to Q320 of SEQ ID NO: 4.

In some embodiments, the UGT includes an amino acid sequence that is at least 95% similar to SEQ ID NO: 5. In some embodiments, the UGT includes an amino acid sequence that is at least 80% identical to a region from V283 to Q323 of SEQ ID NO: 5. In some embodiments, the UGT includes an amino acid sequence that is: at least 90% similar to a region from I67 to Q79 of SEQ ID NO: 5; at least 90% similar to a region from D110 to L118 of SEQ ID NO: 5; at least 90% similar to a region from C131 to T133 of SEQ ID NO: 5; and at least 80% similar to a region from V283 to Q323 of SEQ ID NO: 5.

In some embodiments, the uridine diphosphate-monosaccharide is uridine diphosphate-glucose (“UDP-glucose”), uridine diphosphate-galactose (“UDP-galactose”), uridine diphosphate-xylose (“UDP-xylose”), or uridine diphosphate-N-acetylglucosamine (“UDP-N-acetylglucosamine”).

Described herein are methods of treating cancer. The method can include administering to a patient in need thereof a therapeutically effective amount of a compound having the following structural formula:

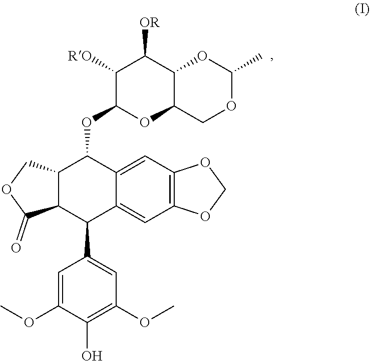

or a pharmaceutically acceptable salt thereof, wherein R and/or R′ is a monosaccharide, a disaccharide, a trisaccharide, or an oligosaccharide comprising 4 to 10 monosaccharides.

In some embodiments, the method further includes administering one or more chemotherapeutic agents (e.g., bevacizumab, bleomycin, carmustine, cisplatin, carboplatin, cyclophosphamide, cytarabine, doxorubicin, ifosfamide, methotrexate, novantrone, procarbazine, thalidomide, vinblastine, and/or vincristine) and/or immune system suppressant (e.g. dexamethasone, prednisone, or methylprednisolone) to the patient.

In some embodiments, the patient has a refractory testicular tumor, small cell lung cancer, lymphoma, non-lymphocytic leukemia, Ewing's sarcoma, Kaposi's sarcoma, a central nervous system cancer, prostate cancer, testicular cancer, ovarian cancer, breast cancer, gastric cancer, or melanoma.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 shows HPLC chromatograms of the UGT screen results using cell lysates from SEQ ID NO: 2 (2: top chromatogram) and empty vector only control (1: bottom chromatogram) when etoposide was used as substrate and UDP-glucose was used as the sugar donor. The two extra peaks highlighted in the chromatogram of SEQ ID NO: 2 were glycosylated products etoposide-3″-O-D-glucoside (chromatogram peak a) and etoposide-4′-O-D-glucoside (chromatogram peak b).

FIG. 2 shows HPLC chromatograms of the purified recombinant glycosyltransferase assay results from SEQ ID NO: 3 (2: middle chromatogram), SEQ ID NO: 2 (3: top chromatogram), and a control reaction containing no glycosyltransferase (1: bottom chromatogram) when etoposide was used as substrate and UDP-glucose was used as the sugar donor. Labeled peaks show etoposide-3″-O-D-glucoside (chromatogram peak a) and etoposide-4′-O-D-glucoside (chromatogram peak b).

FIG. 3 is a chart showing water solubility of etoposide and etoposide-3″-O-D-glucoside.

FIG. 4 is a multiple sequence alignment of five UGTs (SEQ ID NOs: 1-5) highlighting similar sequence regions important for catalytic function. The PSPG box is underlined. The acceptor binding residues are bolded. Sequence Similarity is defined by positive BLAST similarity using the BLOSUM62 scoring matrix and existent: 11, extension: 1 gap penalties.

FIG. 5 shows 3D structures of UGTs indicating the sequence regions that are important for substrate and/or donor binding. All substrates are colored with black carbon sticks (oxygen=red, nitrogen=blue, phosphorus=orange). Cartoon proteins are rainbow from N- to C-terminus. Center: A global structural superposition comprised of multiple UGT crystal structures and homology models. As labeled, zoomed-in regions are clockwise from top-right: I84-S99, L147-S129, A3407-Q382, D126-F134. All numbering follows the sequence of SEQ ID NO: 1 with relevant amino acids shown as sticks.

### DETAILED DESCRIPTION

A description of example embodiments follows.

### Cancer

Cancer is a group of diseases characterized by uncontrolled growth and proliferation of abnormal cells that arises due a combination of genetic and environmental factors. It is the second-leading cause of death worldwide, with cancer causing 1 in 6 deaths each day (American Cancer Society 2018). With the average age of the world population on the rise, the number of new cancer cases is expected to increase.

The 5-year survival rate for cancer patients varies widely depending on many factors including the type of cancer, stage of the cancer at time of diagnosis, patient age, quality of available healthcare, and country of residence. For example, from 2010-2014, the 5-year survival rate for patients with prostate cancer in India was only 44% in comparison to 97% for prostate cancer patients in the United States (American Cancer Society 2018).

The standard cancer treatment regiment typically includes surgery, one or more chemotherapeutic agents, and radiotherapy. Hormone therapy, immunotherapy, and targeted therapies are also possible depending on the characteristics of the cancer. Many additional drugs are often needed to manage the side effects of these treatments.

The monetary cost of cancer comes not only from treatment, cost of care, and rehabilitation, but also from indirect costs, such as loss of work productivity and increased need for home assistance and child care. The cost of cancer worldwide is unknown, but is estimated to be in the hundreds of billions of dollars per year (American Cancer Society 2018). The direct medical cost associated with cancer in the United States in 2015 was approximately $80.2 billion (American Cancer Society 2018).

### Etoposide

Etoposide is a compound represented by the following structural formula:

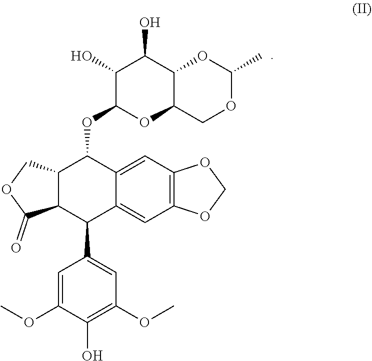

Etoposide (also called VP-16) is a semisynthetic chemotherapeutic first synthesized in 1966 by Sandoz Pharmaceuticals from the natural product podophyllotoxin (Hande 1998). After licensing the drug to Bristol-Myers Squibb in 1978, etoposide was approved by the FDA in 1983 as VePesid to treat various cancers. Etoposide is available as an intravenous (IV) formulation or as an oral capsule to treat refractory testicular tumors, small cell lung cancer, lymphomas, non-lymphocytic leukemia, Ewing's sarcoma, Kaposi's sarcoma, central nervous system cancers, prostate cancer, testicular cancer, and ovarian cancer (Hande 1998; “Etoposide” [2005] 2020; “NCCN Chemotherapy Order Templates” n.d.). Etoposide has also shown some efficacy in breast cancer, gastric cancer, and melanoma in clinical trials (Hande 1998).

While podophyllotoxin binds to microtubules and inhibits its assembly, etoposide only inhibits microtubule assembly at concentrations much higher than that relevant to eliciting a clinical effect. Instead, etoposide exerts its cytotoxic and antitumor activity by poisoning DNA topoisomerase II (Arnold 1979; van Maanen et al. 1988; Hande 1998). DNA topoisomerase II regulates DNA winding and unwinding by temporarily introducing double-stranded breaks in the DNA helix. Etoposide stabilizes the covalently-bound topoisomerase-DNA cleavage complex, resulting in the overaccumulation of transient DNA double-stranded breaks. When other replication machinery or helicases attempt to cross this covalently linked complex, the complex is disrupted, and the double-stranded break becomes permanent. These breaks then undergo recombination and generate insertions, deletions, and chromosomal rearrangements that destabilize the genome and lead to cell death by apoptosis.

The biophysical characteristics and pharmacokinetics/pharmacodynamics (PK/PD) of etoposide has been well described in the decades since its approval (Mylan Pharmaceuticals Inc. 2016; Squibb 2019; Hande 1998). Etoposide is highly lipophilic, with 97% of etoposide bound to blood plasma proteins (primarily albumin). Etoposide undergoes metabolic conversion to secondary metabolites characterized by an open lactone ring, O-demethylation (primarily by the cytochrome P450 CYP3A4), or conjugation by glucuronidation and sulfation. The half-life of etoposide is 4-11 hours. Prolonged exposure to a low dose of etoposide was found to be more therapeutically effective than short-term high doses of etoposide in small cell lung cancer patients. 89% of patients receiving a 5-day etoposide treatment showed a therapeutic response compared to only 10% of patients receiving the same dose of etoposide in 1 day (Slevin et al. 1990).

Despite its success as an anticancer therapeutic, etoposide possesses characteristics that limit its application. Etoposide is highly insoluble in aqueous solutions (150-170 μg/mL at 37° C.), and can only be solubilised in complex formulations containing solubilizers such as polyethylene glycol, Tween 80, and dimethyl sulfoxide (DMSO) (Shah, Chen, and Chow 1989; Hande 1998). Even after solubilizing etoposide and successfully diluting the drug into physiological fluids and commonly used IV formulation diluents, etoposide precipitates after only a few hours at concentrations as low as 1 μg/mL (Tian, He, and Tang 2007; Arnold 1979; Hande 1998). As a result of the low aqueous solubility, treatment with etoposide by IV injection requires large volumes of IV solution. This results in long administration times and restricts the ability of patients to self-administer the drug at home. The requirement for large IV injection volumes and the inclusion of solubilizers results in uncomfortable and dangerous side effects including drug hypersensitivity, hypotension, and heart failure (Hande 1998).

Furthermore, etoposide has a very narrow pH range in which it remains stable (Beijnen et al. 1988). In acidic (pH<5) aqueous environments, etoposide readily loses the C1 sugar moiety. Additional degradative reactions under acidic conditions open the trans-lactone ring to form the hydroxy acid derivative of the etoposide aglycone. In basic (pH>5) aqueous environments, etoposide retains its C1 sugar group but readily undergoes epimerization of the trans-lactone ring to a cis-lactone ring and further degradation to the hydroxy acid derivative. Because the etoposide aglycone and the cis-lactone derivatives exhibit lower cytotoxicities than etoposide, these degradation pathways lead to lower availability of active compound (van Maanen et al. 1988). In addition to this chemical instability, etoposide is a substrate for the drug efflux p-glycoprotein transporter system, further limiting the availability of active etoposide (Squibb 2019).

Oral formulations of etoposide have the benefit of maintaining long-term exposure to low doses of etoposide. However, the inter- and intra-patient variability in bioavailability is markedly high for oral etoposide formulations (ranging from 25% to 50%), perhaps due to the compound's inherent instability and variability in metabolic degradation kinetics in vivo (Toffoli et al. 2001; Rezonja et al. 2013; Squibb 2019; Mylan Pharmaceuticals Inc. 2016; Hande 1998).

Attempts have been made to address these issues. Lipid emulsion formulations containing etoposide have been described that result in a longer shelf life and stability. One example is described by Tian et al (Tian, He, and Tang 2007) in which the shelf life of etoposide in a lipid emulsion is 47 days at 25° C. (compared to 9.5 days in aqueous solutions), and the half life is 54.7 hours at 80° C. and pH 5 (compared to 38.6 minutes in aqueous solutions). However, even in this lipid emulsion formulation, the half-life still decreases significantly with increasing pH (down to 1.5 hours half life at pH 8) and with increasing etoposide concentration.

Several etoposide prodrugs with hydrolyzable moieties at the 4′ hydroxyl group have been described. These hydrolyzable groups include a proponyl carboxyl group, a piperidinopiperidine, a glucose, and a phosphate group (Hatfield et al. 2008; Wrasidlo et al. 2002; Keilholz et al. 2017; US 7,241,595, Kolar et al. 2004; Squibb 2019; Hande 1998). The 4′ hydroxyl group is known to contribute to the bioactivity of etoposide (van Maanen et al. 1988). Thus, prodrugs of etoposide with chemical groups at these positions would be inactive until that chemical group is removed.

The proponyl carboxy etoposide derivatives and the piperidinopiperidine etoposide derivative are converted into active etoposide by carboxyl esterases expressed in various tissues in the body, or by recombinant, bioengineered carboxyl esterases (Hatfield et al. 2008). One proponyl carboxy etoposide derivative called CAP7.1 is cytotoxic to an etoposide-resistant cell line at nanomolar concentrations, and it showed a promising safety profile in a Phase I clinical trial (Keilholz et al. 2017; Wrasidlo et al. 2002). The glycosylated etoposide derivative is described in a patent (U.S. Pat. No. 7,241,595, Kolar et al. 2004) as being hydrolyzed by recombinant glycosidases covalently attached to a tumor-targeting antibody. Etoposide phosphate (Etopophos™) is the one etoposide prodrug derivative that is FDA-approved (Squibb 2019; Hande 1998). Etoposide phosphate is administered by IV injection and is shown to have improved aqueous solubility (20 mg/mL). It is completely and quickly converted to the active form by alkaline dephosphorylases expressed in blood, and it can be safely administered quickly in lower volumes. In preclinical assays and clinical trials, there was no statistically significant difference in PK/PD parameters or overall response rate between treatment with etoposide phosphate plus cisplatin or etoposide plus cisplatin. The main limitation against using this prodrug is cost since the off-patent etoposide is much cheaper.

Other positions on etoposide show promise as potential sites of modification for the development of new etoposide derivatives or prodrugs. For example, the C1 glycoside group, especially the two hydroxyl groups on the glucose, are required for bioactivity (van Maanen et al. 1988). Etoposide prodrugs with modifications at these positions are not believed to have been described thus far. As a result, there are still unexplored opportunities for developing etoposide derivatives or prodrugs with improved aqueous solubility and increased stability. Such a drug derivative may allow the use of more efficient, lower drug doses that could decrease the toxic side effects seen as a result of etoposide's antineoplastic activity and its solubility issues.

### Glycosylation

A potential strategy for improving or modulating the efficacy, safety, and/or PK/PD profile of a small molecule-based therapeutic such as etoposide is modification by glycosylation. The small molecule, or aglycone, is modified by the addition of one or more sugar groups or chains of two or more sugar groups (called oligosaccharides) to nucleophilic centers of the aglycone. These sugar groups can be naturally occurring sugars such as glucose, fructose, rhamnose, mannose, galactose, fucose, xylose, arabinose, glucuronic acid, or N-acetylglucosamine, or they can be synthetically synthesized sugars (e.g., 6-Br-D-glucose, 2-deoxy-D-glucose, 5-thio-D-glucose). These sugars can be attached to the small molecule or to other sugar groups by either an alpha or beta glycosidic bond.

In general, glycosylation of a small molecule can lead to increased aqueous solubility, altered interactions with proteins and membranes, altered absorption and excretion, changes in metabolic stability, and other changes in PK/PD characteristics (Gantt, Peltier-Pain, and Thorson 2011; Křen 2008; De Bruyn et al. 2015).

Glycosylation can enhance or block the transport of a glycoside into specific tissues or organs. Glycosylation can enhance uptake through interaction between the glycoside moiety and lectins or glucose transporters on the cell surface.

In some cases, glycosylation alters the pharmacological activity of the drug, either by enhancing or decreasing potency or even by changing the mechanism of action (Křen 2008; Gantt, Peltier-Pain, and Thorson 2011; De Bruyn et al. 2015).

The identity of the sugar and the stereochemistry of the glycosidic bond can also affect the pharmacological activity or PK/PD profile of a glycoside.

Glycosylation is also a potential strategy for developing prodrugs and compounds for targeted drug delivery to specific tissues. Glycosidases are enzymes that catalyze the hydrolysis of glycosidic bonds and that are specifically expressed in different tissues and organs including blood plasma, the colon, the intestines, and the gut microflora. Glycosidases exhibit substrate specificity towards different glycosidic bond stereochemistry or towards different monosaccharides. A glycosylated drug could function as a prodrug or as a targeted drug if it is preferentially cleaved by a tissue-specific glycosidase. This has been demonstrated by Zipp et al: the alpha-glycosidic bonds in cannabinoid glycosides have been shown to be preferentially cleaved by glycosidases present in the large intestine of mice and not by other chemical or enzymatic processes that may be present in the small intestine, stomach, blood plasma, or brain (Zipp, Hardman, and Brooke 2018; Hardman, Brooke, and Zipp 2017).

An etoposide prodrug modified with a 4′-O-sugar group is reported (U.S. Pat. No. 7,241,595, Kolar et al. 2004). Synthesized by traditional chemical methods, this prodrug is expected to be activated by recombinant glycosidases targeted to tumors by a covalently linked antibody (U.S. Pat. No. 7,241,595, Kolar et al. 2004).

In summary, glycosylation of a small molecule may improve aqueous solubility, but can also alter interactions with proteins and membranes, pharmacological activity, and/or PK/PD characteristics in ways that are unexpected.

### Glycosyltransferases

Traditional methods for glycosylating small molecules are non-selective, and it is particularly difficult to control the stereo- and regiospecificity of glycosylation (Zhu and Schmidt 2009; Gu et al. 2014). There is often more than one position on the aglycone that will react with the reagent used to make the desired modification. This makes it necessary to chemically ‘block’ or render temporarily unreactive, the other positions on the molecule in order to selectively modify the desired position. A typical modification will require multiple protection and de-protection steps using the standard methods of synthetic organic chemistry.

Glycosyltransferases (GTs) are a class of enzymes with the potential to act as the catalyst for the generation of novel glycosylated therapeutic small molecules. GTs catalyze the transfer of a sugar from an activated sugar donor molecule to an acceptor molecule (Lairson et al. 2008). They are a large and well-characterized family found in viruses, archaea, bacteria, and eukaryotes. Greater than 600,000 GTs categorized into approximately 110 families are described in the Carbohydrate-active Enzymes Directory (www.cazy.org), and greater than 150 GT structures are reported (www.rcsb.org) (Lombard et al. 2014; Berman 2000). The majority of GTs utilize nucleotide-activated sugar donors and are referred to as Leloir GTs, although lipid phosphate and phosphate-activated sugar donors are also used (Breton, Fournel-Gigleux, and Palcic 2012; Lairson et al. 2008). GT acceptors include proteins, lipids, oligosaccharides, and small molecules.

GTs offer several advantages as a potential tool in a general small molecule glycosylation platform (De Bruyn et al. 2015; Gantt, Peltier-Pain, and Thorson 2011; Yonekura-Sakakibara and Hanada 2011; Schmid et al. 2016). GTs are often characterized by very high conversion efficiencies (up to 100%). As a result, lower concentrations of potentially expensive or difficult to synthesize substrates are required for GT-catalyzed reactions. GTs are able to glycosylate a wide variety of acceptor structures, with many GTs exhibiting promiscuity towards the sugar donor and acceptor. Furthermore, GTs can catalyze the formation of O-, N-, S-, and even C-glycosides. As a result of these characteristics, GTs are generally amenable to both in vitro and in vivo bioengineering efforts.

### Uridine Diphosphate GTs (UGTs)

Uridine diphosphate GTs (UGTs) utilize uridine diphosphate (UDP) sugar donors, and form the largest group of Leloir GTs in plants (Yonekura-Sakakibara and Hanada 2011). Recently, the identification and characterization of new UGTs, especially in plants and bacteria, has exploded as part of an increased interest in characterizing natural product biosynthetic pathways. This method is described by Torens-Spence et al. (Torrens-Spence et al. 2018). In this paper, 33 UGT enzyme-encoding genes were cloned from a Golden root plant, expressed in yeast, and screened for regiospecific activity in modifying tyrosol to produce salidroside or icariside D2, which are tyrosol metabolites in the plant's native salidroside biosynthetic pathway. Another group identified naturally occurring enzymes having promiscuous N- and O-glycosyltransferase activity by mining the expressed genes of Carthamus tinctorius. K. Xie et al. (Xie et al. 2017) describes the identification of a promiscuous glycosyltransferase (UGT71E5) from C. tinctorius which contains N-glycosylase activity towards multiple diverse nitrogen-heterocyclic aromatic compounds. Zhang et al. (Zhang et al. 2019) describes the identification of three new UGTs (UGT 84A33, UGT 71AE1 and UGT 90A14) from C. tinctorius having promiscuous O-glycosyltransferase activity against benzylisoquinoline alkaloids and their use in making glycosylated derivatives. With the continuing technological improvements and decreasing costs of genome and transcriptome sequencing and analysis, it is becoming easier to identify and characterize naturally occurring GTs for the development of novel small molecule diversity generating platforms.

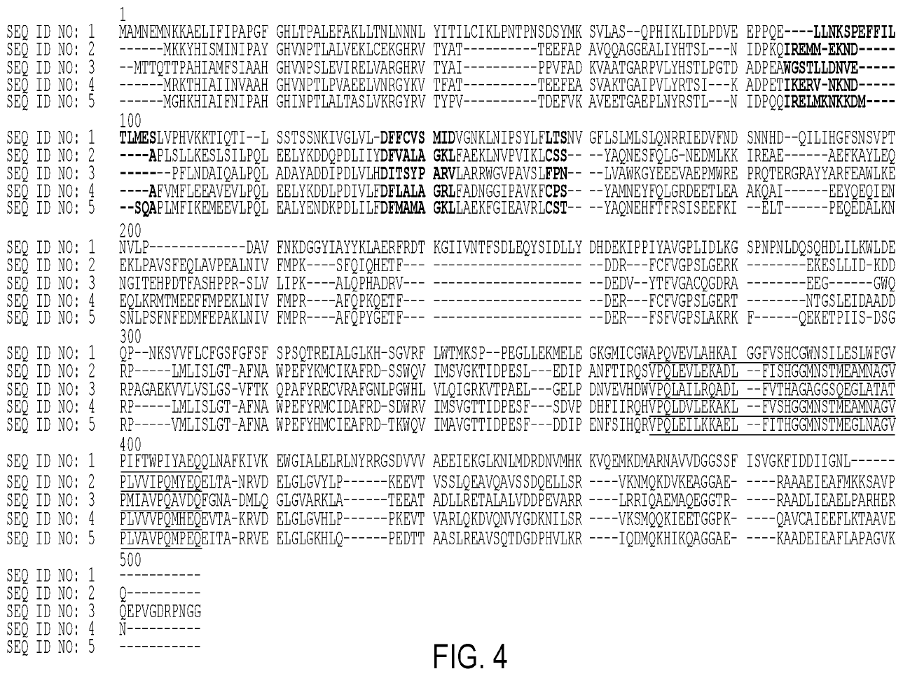

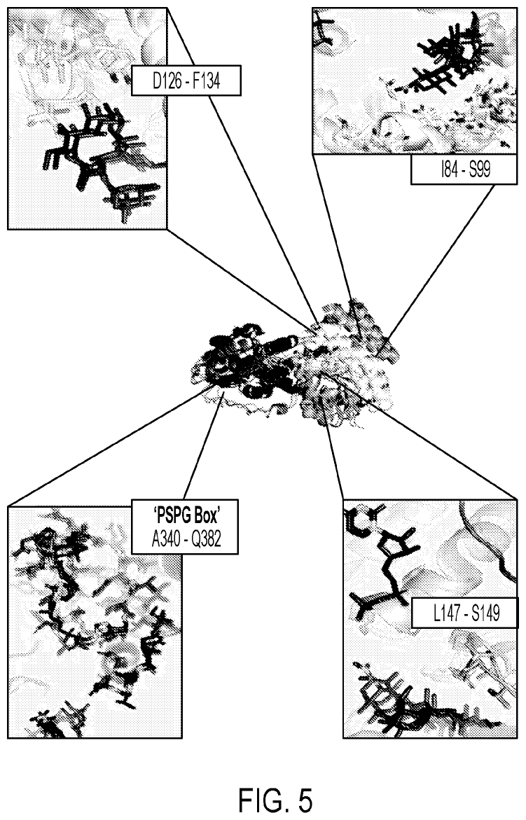

As described herein, four regions within UGT sequences are identified as important for activity. The sequences of all four regions in SEQ ID NO: 1-4 are unique in comparison to other UGTs but highly similar among themselves (FIG. 4). This indicates a strong correlation between the sequences within the four regions and those enzymes' unique activity toward etoposide. Three acceptor binding sites are shown in crystal structures (or homology models) as poised to interact with sugar acceptor molecules. The “PSPG Box” region is involved in both UGT donor and acceptor substrate affinity and is likely a major part of specific activity (FIG. 5) (Bairoch 1991; Hughes and Hughes 1994; Yamazaki, Gong et al. 1999; Hans, Brandt et al. 2004; Shao, He et al. 2005; He, Wang et al. 2006; Offen, Martinez-Fleites et al. 2006).

TABLE 1
UGT Enzyme Regions Important for Activity

| | | | Sequence |
|---|---|---|---|
| Enzyme | Region | Function | Similarity* |
| SEQ ID NO: 1 | I84 - S99 | Acceptor Substrate Binding | 90% |
| (uridine diphosphate | D126 - F134 | Acceptor Substrate Binding | 90% |
| glycosyltransferase | L147 - S149 | Acceptor Substrate Binding | 90% |
| (UGT) from | A340 - Q382 | “PSPG Box” - Donor/Acceptor Binding | 80% |
| Galega orientalis) | | | |
| SEQ ID NO: 2 | I67- D75 | Acceptor Substrate Binding | 90% |
| (uridine diphosphate | D106 - L114 | Acceptor Substrate Binding | 90% |
| glycosyltransferase | C127 - S129 | Acceptor Substrate Binding | 90% |
| (UGT) from | V278 - Q318 | “PSPG Box” - Donor/Acceptor Binding | 80% |
| Bacillus subtilis) | | | |
| SEQ ID NO: 3 | W74 - V82 | Acceptor Substrate Binding | 90% |
| (uridine diphosphate | D111 - V119 | Acceptor Substrate Binding | 90% |
| glycosyltransferase | F132 - N134 | Acceptor Substrate Binding | 90% |
| (UGT) from | V291 - Q331 | “PSPG Box” - Donor/Acceptor Binding | 80% |
| Streptomyces antibioticus) | | | |
| SEQ ID NO: 4 | I67 - D75 | Acceptor Substrate Binding | 90% |
| (uridine diphosphate | D106 - L114 | Acceptor Substrate Binding | 90% |
| glycosyltransferase | C127 - S129 | Acceptor Substrate Binding | 90% |
| (UGT) from | V280 - Q320 | “PSPG Box” - Donor/Acceptor Binding | 80% |
| Bacillus methylotrophicus) | | | |
| SEQ ID NO: 5 | I67 - Q79 | Acceptor Substrate Binding | 90% |
| (uridine diphosphate | D110 - L118 | Acceptor Substrate Binding | 90% |
| glycosyltransferase | C131 - T133 | Acceptor Substrate Binding | 90% |
| (UGT) from | V283 - Q323 | “PSPG Box” - Donor/Acceptor Binding | 80% |
| Bacillus licheniformis) | | | |

* Sequence Similarity is defined by positive BLAST similarity using the BLOSUM62 scoring matrix and existent: 11, extension: 1 gap penalties (Altschul et al. 1990; Henikoff et al. 1992). A commonly used tool for determining percent sequence identity is Protein Basic Local Alignment Search Tool (BLASTp) available through National Center for Biotechnology Information, National Library of Medicine, of the United States National Institutes of Health.

In some embodiments, the UGT includes an amino acid sequence that is at least 80% (85%, 90%, 91%, 92%, 93%, 94%, 95% 96%, 97%, 98%, 99%, or 100%) similar to SEQ ID NO: 1. In some embodiments, the UGT includes an amino acid sequence that is at least 80% (85%, 90%, 91%, 92%, 93%, 94%, 95% 96%, 97%, 98%, 99%, or 100%) identical to SEQ ID NO: 1.

In some embodiments, the UGT includes an amino acid sequence that is at least 80% (85%, 90%, 91%, 92%, 93%, 94%, 95%, 96%, 97%, 98%, 99%, or 100%) similar to a region from A340 to Q382 of SEQ ID NO: 1. In some embodiments, the UGT includes an amino acid sequence that is at least 80% (85%, 90%, 91%, 92%, 93%, 94%, 95%, 96%, 97%, 98%, 99%, or 100%) identical to a region from A340 to Q382 of SEQ ID NO: 1.

In some embodiments, the UGT includes an amino acid sequence that is: at least 90% (91%, 92%, 93%, 94%, 95%, 96%, 97%, 98%, 99%, or 100%) similar to a region from 184 to S99 of SEQ ID NO: 1; at least 90% (91%, 92%, 93%, 94%, 95%, 96%, 97%, 98%, 99%, or 100%) similar to a region from D126 to F134 of SEQ ID NO: 1; at least 90% (91%, 92%, 93%, 94%, 95%, 96%, 97%, 98%, 99%, or 100%) similar to a region from L147 to S149 of SEQ ID NO: 1; and at least 80% (85%, 90%, 91%, 92%, 93%, 94%, 95%, 96%, 97%, 98%, 99%, or 100%) similar to a region from A340 to Q382 of SEQ ID NO: 1.

In some embodiments, the UGT includes an amino acid sequence that is: at least 90% (91%, 92%, 93%, 94%, 95%, 96%, 97%, 98%, 99%, or 100%) identical to a region from 184 to S99 of SEQ ID NO: 1; at least 90% (91%, 92%, 93%, 94%, 95%, 96%, 97%, 98%, 99%, or 100%) identical to a region from D126 to F134 of SEQ ID NO: 1; at least 90% (91%, 92%, 93%, 94%, 95%, 96%, 97%, 98%, 99%, or 100%) identical to a region from L147 to S149 of SEQ ID NO: 1; and at least 80% (85%, 90%, 91%, 92%, 93%, 94%, 95%, 96%, 97%, 98%, 99%, or 100%) identical to a region from A340 to Q382 of SEQ ID NO: 1.

In some embodiments, the UGT includes an amino acid sequence that is at least 80% (85%, 90%, 91%, 92%, 93%, 94%, 95% 96%, 97%, 98%, 99%, or 100%) similar to SEQ ID NO: 2. In some embodiments, the UGT includes an amino acid sequence that is at least 80% (85%, 90%, 91%, 92%, 93%, 94%, 95% 96%, 97%, 98%, 99%, or 100%) identical to SEQ ID NO: 2.

In some embodiments, the UGT includes an amino acid sequence that is at least 80% (85%, 90%, 91%, 92%, 93%, 94%, 95%, 96%, 97%, 98%, 99%, or 100%) similar to a region from V278 to Q318 of SEQ ID NO: 2. In some embodiments, the UGT includes an amino acid sequence that is at least 80% (85%, 90%, 91%, 92%, 93%, 94%, 95%, 96%, 97%, 98%, 99%, or 100%) identical to a region from V278 to Q318 of SEQ ID NO: 2.

In some embodiments, the UGT includes an amino acid sequence that is: at least 90% (91%, 92%, 93%, 94%, 95%, 96%, 97%, 98%, 99%, or 100%) similar to a region from I67 to D75 of SEQ ID NO: 2; at least 90% (91%, 92%, 93%, 94%, 95%, 96%, 97%, 98%, 99%, or 100%) similar to a region from D106 to L114 of SEQ ID NO: 2; at least 90% (91%, 92%, 93%, 94%, 95%, 96%, 97%, 98%, 99%, or 100%) similar to a region from C127 to S129 of SEQ ID NO: 2; and at least 80% (85%, 90%, 91%, 92%, 93%, 94%, 95%, 96%, 97%, 98%, 99%, or 100%) similar to a region from V278 to Q318 of SEQ ID NO: 2.

In some embodiments, the UGT includes an amino acid sequence that is: at least 90% (91%, 92%, 93%, 94%, 95%, 96%, 97%, 98%, 99%, or 100%) identical to a region from I67 to D75 of SEQ ID NO: 2; at least 90% (91%, 92%, 93%, 94%, 95%, 96%, 97%, 98%, 99%, or 100%) identical to a region from D106 to L114 of SEQ ID NO: 2; at least 90% (91%, 92%, 93%, 94%, 95%, 96%, 97%, 98%, 99%, or 100%) identical to a region from C127 to S129 of SEQ ID NO: 2; and at least 80% (85%, 90%, 91%, 92%, 93%, 94%, 95%, 96%, 97%, 98%, 99%, or 100%) identical to a region from V278 to Q318 of SEQ ID NO: 2.

In some embodiments, the UGT includes an amino acid sequence that is at least 80% (85%, 90%, 91%, 92%, 93%, 94%, 95% 96%, 97%, 98%, 99%, or 100%) similar to SEQ ID NO: 3. In some embodiments, the UGT includes s an amino acid sequence that is at least 80% (85%, 90%, 91%, 92%, 93%, 94%, 95% 96%, 97%, 98%, 99%, or 100%) identical to SEQ ID NO: 3.

In some embodiments, the UGT includes an amino acid sequence that is at least 80% (85%, 90%, 91%, 92%, 93%, 94%, 95%, 96%, 97%, 98%, 99%, or 100%) similar to a region from V291 to Q331 of SEQ ID NO: 3. In some embodiments, the UGT includes an amino acid sequence that is at least 80% (85%, 90%, 91%, 92%, 93%, 94%, 95%, 96%, 97%, 98%, 99%, or 100%) identical to a region from V291 to Q331 of SEQ ID NO: 3.

In some embodiments, the UGT includes an amino acid sequence that is: at least 90% (91%, 92%, 93%, 94%, 95%, 96%, 97%, 98%, 99%, or 100%) similar to a region from W74 to V82 of SEQ ID NO: 3; at least 90% (91%, 92%, 93%, 94%, 95%, 96%, 97%, 98%, 99%, or 100%) similar to a region from D111 to V119 of SEQ ID NO: 3; at least 90% (91%, 92%, 93%, 94%, 95%, 96%, 97%, 98%, 99%, or 100%) similar to a region from F132 to N134 of SEQ ID NO: 3; and at least 80% (85%, 90%, 91%, 92%, 93%, 94%, 95%, 96%, 97%, 98%, 99%, or 100%) similar to a region from V291 to Q331 of SEQ ID NO: 3.

In some embodiments, the UGT includes an amino acid sequence that is: at least 90% (91%, 92%, 93%, 94%, 95%, 96%, 97%, 98%, 99%, or 100%) identical to a region from W74 to V82 of SEQ ID NO: 3; at least 90% (91%, 92%, 93%, 94%, 95%, 96%, 97%, 98%, 99%, or 100%) identical to a region from D111 to V119 of SEQ ID NO: 3; at least 90% (91%, 92%, 93%, 94%, 95%, 96%, 97%, 98%, 99%, or 100%) identical to a region from F132 to N134 of SEQ ID NO: 3; and at least 80% (85%, 90%, 91%, 92%, 93%, 94%, 95%, 96%, 97%, 98%, 99%, or 100%) identical to a region from V291 to Q331 of SEQ ID NO: 3.

In some embodiments, the UGT includes an amino acid sequence that is at least 80% (85%, 90%, 91%, 92%, 93%, 94%, 95% 96%, 97%, 98%, 99%, or 100%) similar to SEQ ID NO: 4. In some embodiments, the UGT includes an amino acid sequence that is at least 80% (85%, 90%, 91%, 92%, 93%, 94%, 95% 96%, 97%, 98%, 99%, or 100%) identical to SEQ ID NO: 4.

In some embodiments, the UGT includes an amino acid sequence that is at least 80% (85%, 90%, 91%, 92%, 93%, 94%, 95%, 96%, 97%, 98%, 99%, or 100%) similar to a region from V280 to Q320 of SEQ ID NO: 4. In some embodiments, the UGT includes an amino acid sequence that is at least 80% (85%, 90%, 91%, 92%, 93%, 94%, 95%, 96%, 97%, 98%, 99%, or 100%) identical to a region from V280 to Q320 of SEQ ID NO: 4.

In some embodiments, the UGT includes an amino acid sequence that is: at least 90% (91%, 92%, 93%, 94%, 95%, 96%, 97%, 98%, 99%, or 100%) similar to a region from I67 to D75 of SEQ ID NO: 4; at least 90% (91%, 92%, 93%, 94%, 95%, 96%, 97%, 98%, 99%, or 100%) similar to a region from D106 to L114 of SEQ ID NO: 4; at least 90% (91%, 92%, 93%, 94%, 95%, 96%, 97%, 98%, 99%, or 100%) similar to a region from C127 to S129 of SEQ ID NO: 4; and at least 80% (85%, 90%, 91%, 92%, 93%, 94%, 95%, 96%, 97%, 98%, 99%, or 100%) similar to a region from V280 to Q320 of SEQ ID NO: 4.

In some embodiments, the UGT includes an amino acid sequence that is: at least 90% (91%, 92%, 93%, 94%, 95%, 96%, 97%, 98%, 99%, or 100%) identical to a region from I67 to D75 of SEQ ID NO: 4; at least 90% (91%, 92%, 93%, 94%, 95%, 96%, 97%, 98%, 99%, or 100%) identical to a region from D106 to L114 of SEQ ID NO: 4; at least 90% (91%, 92%, 93%, 94%, 95%, 96%, 97%, 98%, 99%, or 100%) identical to a region from C127 to S129 of SEQ ID NO: 4; and at least 80% (85%, 90%, 91%, 92%, 93%, 94%, 95%, 96%, 97%, 98%, 99%, or 100%) identical to a region from V280 to Q320 of SEQ ID NO: 4.

In some embodiments, the UGT includes an amino acid sequence that is at least 80% (85%, 90%, 91%, 92%, 93%, 94%, 95% 96%, 97%, 98%, 99%, or 100%) similar to SEQ ID NO: 5. In some embodiments, the UGT includes an amino acid sequence that is at least 80% (85%, 90%, 91%, 92%, 93%, 94%, 95% 96%, 97%, 98%, 99%, or 100%) identical to SEQ ID NO: 5.

In some embodiments, the UGT includes an amino acid sequence that is at least 80% (85%, 90%, 91%, 92%, 93%, 94%, 95%, 96%, 97%, 98%, 99%, or 100%) similar to a region from V283 to Q323 of SEQ ID NO: 5. In some embodiments, the UGT includes an amino acid sequence that is at least 80% (85%, 90%, 91%, 92%, 93%, 94%, 95%, 96%, 97%, 98%, 99%, or 100%) identical to a region from V283 to Q323 of SEQ ID NO: 5.

In some embodiments, the UGT includes an amino acid sequence that is: at least 90% (91%, 92%, 93%, 94%, 95%, 96%, 97%, 98%, 99%, or 100%) similar to a region from I67 to Q79 of SEQ ID NO: 5; at least 90% (91%, 92%, 93%, 94%, 95%, 96%, 97%, 98%, 99%, or 100%) similar to a region from D110 to L118 of SEQ ID NO: 5; at least 90% (91%, 92%, 93%, 94%, 95%, 96%, 97%, 98%, 99%, or 100%) similar to a region from C131 to T133 of SEQ ID NO: 5; and at least 80% (85%, 90%, 91%, 92%, 93%, 94%, 95%, 96%, 97%, 98%, 99%, or 100%) similar to a region from V283 to Q323 of SEQ ID NO: 5.

In some embodiments, the UGT includes an amino acid sequence that is: at least 90% (91%, 92%, 93%, 94%, 95%, 96%, 97%, 98%, 99%, or 100%) identical to a region from I67 to Q79 of SEQ ID NO: 5; at least 90% (91%, 92%, 93%, 94%, 95%, 96%, 97%, 98%, 99%, or 100%) identical to a region from D110 to L118 of SEQ ID NO: 5; at least 90% (91%, 92%, 93%, 94%, 95%, 96%, 97%, 98%, 99%, or 100%) identical to a region from C131 to S133 of SEQ ID NO: 5; and at least 80% (85%, 90%, 91%, 92%, 93%, 94%, 95%, 96%, 97%, 98%, 99%, or 100%) identical to a region from V283 to Q323 of SEQ ID NO: 5.

### Monosaccharides, Disaccharides, Trisaccharides, and Oligosaccharides

Glycosyltransferases can catalyze the addition of many different monosaccharides to etoposide. In general, suitable monosaccharides include, but are not limited to, open and closed chain monosaccharides. The monosaccharides can be in the L- or D-configuration. Typically, the monosaccharides have 5, 6, or 7 carbons (a pentose monosaccharide, hexose monosaccharide, or heptose monosaccharide, respectively).

Suitable monosaccharides include allose, apiose, arabinose, fructose, fucitol, fucose, galactose, glucose, glucuronic acid, mannose, N-acetylglucosamine, N-acetylgalactosamine, rhamnose, and xylose. Other suitable monosaccharides include glucosamine, galactosamine, mannosamine, 5-thio-D-glucose, nojirimycin, deoxynojirimycin, 1,5-anhydro-D-sorbitol, 2,5-anhydro-D-mannitol, 2-deoxy-D-galactose, 2-deoxy-D-glucose, 3-deoxy-D-glucose, arabinitol, galactitol, glucitol, iditol, lyxose, mannitol, L-rhamnitol, 2-deoxy-D-ribose, ribose, ribitol, ribulose, xylulose, altrose, gulose, idose, levulose, psicose, sorbose, tagatose, talose, galactal, glucal, fucal, rhamnal, arabinal, xylal, 3,4-di-O-acetyl-L-fucal, 3,4-di-O-acetyl-L-rhamnal, 3,4-di-O-acetyl-D-arabinal, 3,4-di-O-acetyl-D-xylal, valienamine, validamine, valiolamine, valienol, valienone, galacturonic acid, mannuronic acid, N-acetylneuraminic acid, N-acetylmuramic acid, gluconic acid D-lactone, galactonic acid gamma-lactone, galactonic acid delta-lactone, mannonic acid gamma-lactone, D-altro-heptulose, D-manno-heptulose, D-glycero-D-manno-heptose, D-glycero-D-gluco-heptose, D-allo-heptulose, D-altro-3-heptulose, D-glycero-D-manno-heptitol, and D-glycero-D-altro-heptitol.

Suitable oligosaccharides include, but are not limited to, carbohydrates having from 2 to 10 or more monosaccharides linked together (e.g., 2, 3, 4, 5, 6, 7, 8, 9, or 10 monosaccharides linked together). The constituent monosaccharide unit may be, for example, a pentose monosaccharide, a hexose monosaccharide, or a pseudosugar (including a pseudoamino sugar). Oligosaccharides do not include bicyclic groups that are formed by fusing a monosaccharide to a benzene ring, a cyclohexane ring, or a heterocyclic ring. Pseudosugars that may be used in the invention are members of the class of compounds wherein the ring oxygen atom of the cyclic monosaccharide is replaced by a methylene group. Pseudosugars are also known as “carba-sugars.”

The glycosyltransferases can catalyze addition of a monosaccharide to etoposide, and the bond between the monosaccharide and etoposide can be either an alpha or beta glycosidic bond. Disaccharides, trisaccharides, and oligosaccharides are formed by serial enzymatic additions of two or more monosaccharides to etoposide. When more than one monosaccharide is added by serial enzymatic reactions, successive monosaccharides can be bonded to the preceding monosaccharide by either an alpha or beta glycosidic bond.

### Methods of Making Etoposide Glycosides

Etoposide glycosides can be made from etoposide by an enzymatically catalyzed reaction. A reaction mixture is provided that includes etoposide, a uridine diphosphate glycosyltransferase, and a uridine diphosphate-monosaccharide. After a period of time (e.g., from 1 to 72 hours), etoposide is converted to a monosaccharide, disaccharide, trisaccharide, or oligosaccharide of etoposide. The monosaccharide, disaccharide, trisaccharide, or oligosaccharide of etoposide that is formed corresponds to the uridine diphosphate-monosaccharide that is included in the reaction mixture.

In some embodiments, the UGT enzyme and recombinant UGT-expressing cell lysate (e.g., yeast cell lysate) are placed in a reaction vessel. To form the lysate, UGT-expressing cells (e.g., UGT-expressing yeast cells) are lysed and the insoluble part is discarded by centrifugation so that the lysate is cell-free. In other embodiments, the cell-free lysate is not required. For example, in some embodiments, recombinant UGTs can be used. In other embodiments, purified UGTs can be used.

### Etoposide Glycosides

In some embodiments, etoposide glycosides are compounds represented by the following structural formula:

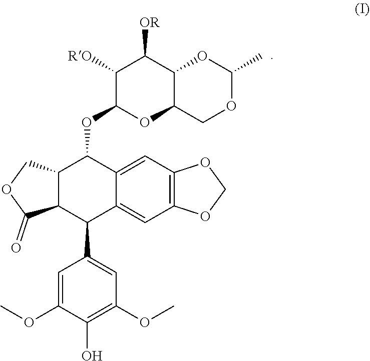

R and/or R′ is a hydrogen, a monosaccharide, a disaccharide, a trisaccharide, or an oligosaccharide. The oligosaccharide can include 4 to 10 monosaccharides (e.g. 4, 5, 6, 7, 8, 9, or 10 monosaccharides). Each of R and R′ can independently be a monosaccharide, a disaccharide, or an oligosaccharide. In some instances, the compound is a pharmaceutically acceptable salt of Compound (I).

In some embodiments, etoposide glycosides are compounds represented by the following structural formula:

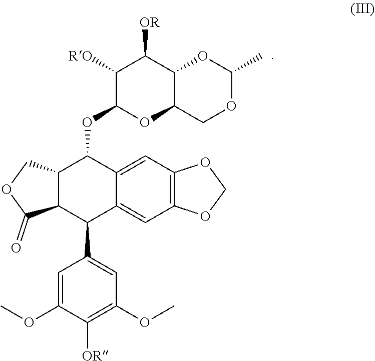

R, R′, and/or R″ is a hydrogen, a monosaccharide, a disaccharide, a trisaccharide, or an oligosaccharide comprising 4 to 10 monosaccharides (e.g. 4, 5, 6, 7, 8, 9, or 10 monosaccharides). Each of R, R′, and R″ can independently be a monosaccharide, a disaccharide, or an oligosaccharide. In some instances, the compound is a pharmaceutically acceptable salt of Compound (II).

In some embodiments, R″ is not glucose.

In one embodiment, R, R′, and/or R″ is glucose, which can be D-glucose or L-glucose. D-glucose is represented by the following structural formula:

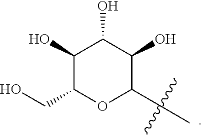 [SMILES: *C1O[C@H](CO)[C@@H](O)[C@H](O)[C@H]1O]

In one embodiment, R, R′, and/or R″ is galactose, which can be D-galactose or L-galactose. D-galactose is represented by the following structural formula:

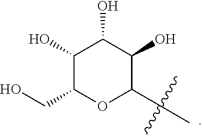 [SMILES: *C1O[C@H](CO)[C@H](O)[C@H](O)[C@H]1O]

In one embodiment, R, R′, and/or R″ is xylose, which can be D-xylose or L-xylose. Xylose can form six- and five-membered rings. A five-membered ring of D-xylose is represented by the following structural formula:

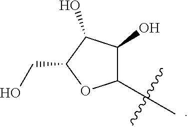 [SMILES: *C1O[C@H](CO)[C@H](O)[C@H]1O]

In one embodiment, R, R′, and/or R″ is N-acetylglucosamine, which can be D-N-acetylglucosamine or L-N-acetylglucosamine. D-N-acetylglucosamine is represented by the following structural formula:

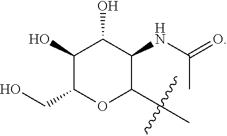 [SMILES: *C1O[C@H](CO)[C@@H](O)[C@H](O)[C@H]1NC(C)=O]

The bond between the monosaccharide (e.g., glucose) and etoposide can be an alpha or beta glycosidic bond. The bond between monosaccharides of a disaccharide can be either an alpha or beta glycosidic bond. The bond between monosaccharides of a trisaccharide can be either an alpha or beta glycosidic bond. The bond between monosaccharides of an oligosaccharide can be either an alpha or beta glycosidic bond. The glycosidic bond between monosaccharides of a disaccharide or a trisaccharide and between monosaccharides of an oligosaccharide can be formed between any of the hydroxyl groups from each monosaccharide. In other words, the bond between monosaccharides can be, e.g., 1→2, 1∝3, 1→4, or 1→6.

In some embodiments, R, R′, and/or R″ is a disaccharide.

In one embodiment, R, R′, and/or R″ is a disaccharide consisting of two molecules of glucose. One example is etoposide-3″-di-O-D-glucoside. Another example is etoposide-4′-di-O-D-glucoside. A disaccharide consisting of two monomers of glucose, where the two monomers are bonded by a 1→2 glycosidic bond, has the following structural formula:

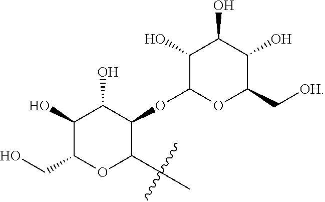 [SMILES: *C1O[C@H](CO)[C@@H](O)[C@H](O)[C@H]1OC1O[C@H](CO)[C@@H](O)[C@H](O)[C@H]1O]

In one embodiment, R, R′, and/or R″ is a disaccharide consisting of two molecules of galactose. One example is etoposide-3″-di-O-D-galactoside. Another example is etoposide-4′-di-O-D-galactoside. A disaccharide consisting of two monomers of galactose, where the two monomers are bonded by a 1→2 glycosidic bond, has the following structural formula:

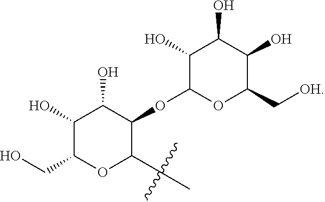 [SMILES: *C1O[C@H](CO)[C@H](O)[C@H](O)[C@H]1OC1O[C@H](CO)[C@H](O)[C@H](O)[C@H]1O]

In one particular embodiment, R, R′, and/or R″ is a disaccharide consisting of two molecules of xylose. One example is etoposide-3″-di-O-D-xyloside. Another example is etoposide-4′-di-O-D-xyloside. A disaccharide consisting of two monomers of xylose, where the two monomers are bonded by a 1→2 glycosidic bond, has the following structural formula:

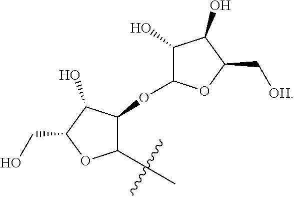 [SMILES: *C1O[C@H](CO)[C@H](O)[C@H]1OC1O[C@H](CO)[C@H](O)[C@H]1O]

In some embodiments, the disaccharide includes two different monosaccharides. In some embodiments, the oligosaccharide includes two or more different monosaccharides. One example is etoposide-3″-O-xylose-glucoside. Another example is etoposide-4′-O-xylose-glucoside.

### Methods of Treating Diseases

The etoposide glycosides described herein can be used in methods of treating diseases. The etoposide glycoside is administered to a patient in need thereof.

Diseases that can be treated by administering the etoposide glycosides disclosed herein include, but are not limited to, cancer, such as a refractory testicular tumor, small cell lung cancer, lymphoma, non-lymphocytic leukemia, Ewing's sarcoma, Kaposi's sarcoma, ovarian cancer, a central nervous system cancer, prostate cancer, testicular cancer, breast cancer, gastric cancer, and melanoma.

The etoposide glycosides can be administered as part of a combination therapy.

One example of a combination therapy is administration with cisplatin. Other examples include administration with one or more of a chemotherapeutic agent (e.g., bevacizumab, bleomycin, carmustine, cisplatin, carboplatin, cyclophosphamide, cytarabine, doxorubicin, ifosfamide, methotrexate, novantrone, procarbazine, thalidomide, vinblastine, and/or vincristine) and/or immune system suppressant (e.g. dexamethasone, prednisone, or methylprednisolone).

The etoposide glycosides described herein can be used in place of, or in addition to, etoposide in those combination therapies.

### Pharmaceutical Compositions, Dosing, and Administration

Also provided herein is a pharmaceutical composition, comprising an etoposide glycoside disclosed herein, or a pharmaceutically acceptable salt thereof, and optionally a pharmaceutically acceptable carrier. The compositions can be used in the methods described herein, e.g., to supply a compound described herein, or a pharmaceutically acceptable salt thereof

“Pharmaceutically acceptable salt” refers to those salts which are, within the scope of sound medical judgment, suitable for use in contact with the tissues of mammals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, the relevant teachings of which are incorporated herein by reference in their entirety. Pharmaceutically acceptable salts of the compounds described herein include salts derived from suitable inorganic and organic acids, and suitable inorganic and organic bases.

Examples of pharmaceutically acceptable acid addition salts are salts of an amino group formed with inorganic acids such as hydrochloric acid, hydrobromic acid, phosphoric acid, sulfuric acid and perchloric acid, or with organic acids such as acetic acid, oxalic acid, maleic acid, tartaric acid, citric acid, succinic acid or malonic acid or by using other methods used in the art, such as ion exchange. Other pharmaceutically acceptable acid addition salts include adipate, alginate, ascorbate, aspartate, benzenesulfonate, benzoate, bisulfate, borate, butyrate, camphorate, camphorsulfonate, cinnamate, citrate, cyclopentanepropionate, digluconate, dodecylsulfate, ethanesulfonate, formate, fumarate, glucoheptonate, glycerophosphate, gluconate, glutarate, glycolate, hemisulfate, heptanoate, hexanoate, hydroiodide, hydroxybenzoate, 2-hydroxy-ethanesulfonate, hydroxymaleate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 2-phenoxybenzoate, phenylacetate, 3-phenylpropionate, phosphate, pivalate, propionate, pyruvate, salicylate, stearate, succinate, sulfate, tartrate, thiocyanate, p-toluenesulfonate, undecanoate, valerate salts, and the like.

Either the mono-, di- or tri-acid salts can be formed, and such salts can exist in either a hydrated, solvated or substantially anhydrous form.

Salts derived from appropriate bases include salts derived from inorganic bases, such as alkali metal, alkaline earth metal, and ammonium bases, and salts derived from aliphatic, alicyclic or aromatic organic amines, such as methylamine, trimethylamine and picoline, or N⁺((C₁-C₄)alkyl)₄ salts. Representative alkali or alkaline earth metal salts include sodium, lithium, potassium, calcium, magnesium, barium and the like. Further pharmaceutically acceptable salts include, when appropriate, nontoxic ammonium, quaternary ammonium, and amine cations formed using counterions such as halide, hydroxide, carboxyl, sulfate, phosphate, nitrate, lower alkyl sulfonate and aryl sulfonate.

“Pharmaceutically acceptable carrier” refers to a non-toxic carrier or excipient that does not destroy the pharmacological activity of the agent with which it is formulated and is nontoxic when administered in doses sufficient to deliver a therapeutic amount of the agent. Pharmaceutically acceptable carriers that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions provided herein can be orally administered in any orally acceptable dosage form including, but not limited to, capsules, tablets, aqueous suspensions, dispersions and solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions and/or emulsions are required for oral use, the active ingredient can be suspended or dissolved in an oily phase and combined with emulsifying and/or suspending agents. If desired, certain sweetening, flavoring or coloring agents may also be added.

In some embodiments, an oral formulation is formulated for immediate release or sustained/delayed release.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders, such as carboxymethylcellulose, alginates, gelatin, polyvinylpyrrolidinone, sucrose, and acacia, (c) humectants such as glycerol, (d) disintegrating agents such as agar-agar, calcium carbonate, potato or tapioca starch, alginic acid, certain silicates, and sodium carbonate, (e) solution retarding agents such as paraffin, (f) absorption accelerators such as quaternary ammonium salts, (g) wetting agents, such as acetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Liquid dosage forms for oral administration include pharmaceutically acceptable emulsions, microemulsions, solutions, suspensions, syrups, and elixirs. In addition to the etoposide glycosides of the present disclosure, the liquid dosage forms may contain inert diluents commonly used in the art, such as water or other solvents, solubilizing agents and emulsifiers, such as ethyl alcohol (ethanol), isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, or mixtures thereof. Besides inert diluents, the oral compositions can also include adjuvants such as wetting agents, emulsifying and suspending agents, sweetening, flavoring, coloring, perfuming, and preservative agents.

Compositions suitable for buccal or sublingual administration include tablets, lozenges and pastilles, wherein the active ingredient is formulated with a carrier such as sugar and acacia, tragacanth, or gelatin and glycerin.

Solid compositions of a similar type may also be employed as fillers in soft and hard-filled gelatin capsules using excipients such as lactose or milk sugar, as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

An etoposide glycoside described herein can also be in micro-encapsulated form with one or more excipients, as noted above. In such solid dosage forms, the etoposide glycoside can be admixed with at least one inert diluent such as sucrose, lactose or starch. Such dosage forms can also comprise, as is normal practice, additional substances other than inert diluents, e.g., tableting lubricants and other tableting aids such a magnesium stearate and microcrystalline cellulose.

Compositions for oral administration may be designed to protect the active ingredient against degradation as it passes through the alimentary tract, for example, by an outer coating of the formulation on a tablet or capsule.

In another embodiment, an etoposide glycoside or pharmaceutically acceptable salt described herein can be provided in an extended (or “delayed” or “sustained”) release composition. This delayed-release composition includes the etoposide glycoside or pharmaceutically acceptable salt in combination with a delayed-release component. Such a composition allows targeted release of a provided agent into the lower gastrointestinal tract, for example, into the small intestine, the large intestine, the colon and/or the rectum. In certain embodiments, a delayed-release composition further includes an enteric or pH-dependent coating, such as cellulose acetate phthalates and other phthalates (e.g., polyvinyl acetate phthalate, methacrylates (Eudragits)). Alternatively, the delayed-release composition provides controlled release to the small intestine and/or colon by the provision of pH sensitive methacrylate coatings, pH sensitive polymeric microspheres, or polymers which undergo degradation by hydrolysis. The delayed-release composition can be formulated with hydrophobic or gelling excipients or coatings. Colonic delivery can further be provided by coatings which are digested by bacterial enzymes such as amylose or pectin, by pH dependent polymers, by hydrogel plugs swelling with time (Pulsincap), by time-dependent hydrogel coatings and/or by acrylic acid linked to azoaromatic bonds coatings.

The amount of an etoposide glycoside described herein, or a pharmaceutically acceptable salt thereof, that can be combined with the carrier materials to produce a composition in a single dosage form will vary depending upon the host treated, the particular mode of administration and the activity of the agent employed. Preferably, compositions should be formulated so that a dosage of from about 0.01 mg/kg to about 100 mg/kg body weight/day of the etoposide glycoside, or pharmaceutically acceptable salt thereof, can be administered to a subject receiving the composition.

The desired dose may conveniently be administered in a single dose or as multiple doses administered at appropriate intervals such that, for example, the agent is administered 2, 3, 4, 5, 6 or more times per day. The daily dose can be divided, especially when relatively large amounts are administered, or as deemed appropriate, into several, for example 2, 3, 4, 5, 6 or more, administrations.

It should also be understood that a specific dosage and treatment regimen for any particular patient will depend upon a variety of factors, including the activity of the specific agent employed, the age, body weight, general health, sex, diet, time of administration, rate of excretion, drug combination, the judgment of the treating physician and the severity of the particular disease being treated. The amount of an etoposide glycoside in the composition will also depend upon the particular etoposide glycoside in the composition.

Other pharmaceutically acceptable carriers, adjuvants and vehicles that can be used in the compositions of this invention include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives can also be advantageously used to enhance delivery of agents described herein.

In some embodiments, compositions comprising an etoposide glycoside described herein, or a pharmaceutically acceptable salt thereof, can also include one or more other therapeutic agents, e.g., in combination. When the compositions of this invention comprise a combination, the agents should be present at dosage levels of between about 1 to 100%, and more preferably between about 5% to about 95% of the dosage normally administered in a monotherapy regimen.

The compositions described herein can, for example, be administered by injection, intravenously, intraarterially, intraocularly, intravitreally, subdermally, orally, buccally, nasally, transmucosally, topically, in an ophthalmic preparation, or by inhalation, with a dosage ranging from about 0.5 mg/kg to about 100 mg/kg of body weight or, alternatively, in a dosage ranging from about 1 mg/dose to about 1000 mg/dose, every 4 to 120 hours, or according to the requirements of the particular drug. Typically, the compositions will be administered from about 1 to about 6 (e.g., 1, 2, 3, 4, 5 or 6) times per day or, alternatively, as an infusion (e.g., a continuous infusion). The amount of active ingredient that can be combined with a carrier material to produce a single dosage form will vary depending upon the host treated and the particular mode of administration. A typical preparation will contain from about 1% to about 95%, from about 2.5% to about 95% or from about 5% to about 95% of an etoposide glycoside (w/w). Alternatively, a preparation can contain from about 20% to about 80% of an etoposide glycoside (w/w).

Doses lower or higher than those recited above may be required. Specific dosage and treatment regimens for any particular patient will depend upon a variety of factors, including the activity of the specific agent employed, the age, body weight, general health status, sex, diet, time of administration, rate of excretion, drug combination, the severity and course of the disease, condition or symptoms, the patient's disposition to the disease, condition or symptoms, and the judgment of the treating physician.

“Treating,” as used herein, refers to taking steps to deliver a therapy to a subject, such as a mammal, in need thereof (e.g., as by administering to a mammal one or more therapeutic agents). “Treating” includes inhibiting the disease or condition (e.g., as by slowing or stopping its progression or causing regression of the disease or condition), and relieving the symptoms resulting from the disease or condition.

“A therapeutically effective amount” is an amount effective, at dosages and for periods of time necessary, to achieve a desired therapeutic result (e.g., treatment, healing, inhibition or amelioration of physiological response or condition, etc.). The full therapeutic effect does not necessarily occur by administration of one dose, and may occur only after administration of a series of doses. Thus, a therapeutically effective amount may be administered in one or more administrations. A therapeutically effective amount may vary according to factors such as disease state, age, sex, and weight of a mammal, mode of administration and the ability of a therapeutic, or combination of therapeutics, to elicit a desired response in an individual.

An effective amount of an agent to be administered can be determined by a clinician of ordinary skill using the guidance provided herein and other methods known in the art. For example, suitable dosages can be from about 0.001 mg/kg to about 100 mg/kg, from about 0.01 mg/kg to about 100 mg/kg, from about 0.01 mg/kg to about 10 mg/kg, from about 0.01 mg/kg to about 1 mg/kg body weight per treatment. Determining the dosage for a particular agent, subject and disease is well within the abilities of one of skill in the art. Preferably, the dosage does not cause adverse side effects or produces minimal adverse side effects.

As used herein, “subject” includes humans, domestic animals, such as laboratory animals (e.g., dogs, monkeys, pigs, rats, mice, etc.), household pets (e.g., cats, dogs, rabbits, etc.) and livestock (e.g., pigs, cattle, sheep, goats, horses, etc.), and non-domestic animals. In some embodiments, a subject is a human. “Subject” and “patient” are used interchangeably herein.

An etoposide glycoside described herein, or a pharmaceutically acceptable salt thereof, can be administered via a variety of routes of administration, including, for example, oral, dietary, topical, transdermal, rectal, parenteral (e.g., intra-arterial, intravenous, intramuscular, subcutaneous injection, intradermal injection), intravenous infusion and inhalation (e.g., intrabronchial, intranasal or oral inhalation, intranasal drops) routes of administration, depending on the etoposide glycoside and the particular disease to be treated. Administration can be local or systemic as indicated. The preferred mode of administration can vary depending on the particular etoposide glycoside chosen.

Certain methods further specify a delivery route such as intravenous, intramuscular, subcutaneous, rectal, intranasal, pulmonary, or oral.

An etoposide glycoside described herein, or a pharmaceutically acceptable salt thereof, can also be administered in combination with one or more other therapies (e.g., radiation therapy, a chemotherapy, such as a chemotherapeutic agent; an immunotherapy, such as an immunotherapeutic agent). When administered in a combination therapy, the etoposide glycoside, or pharmaceutically acceptable salt thereof, can be administered before, after or concurrently with the other therapy (e.g., radiation therapy, an additional agent(s)). When co-administered simultaneously (e.g., concurrently), the etoposide glycoside, or pharmaceutically acceptable salt thereof, and other therapy can be in separate formulations or the same formulation. Alternatively, the etoposide glycoside, or pharmaceutically acceptable salt thereof, and other therapy can be administered sequentially, as separate compositions, within an appropriate time frame as determined by a skilled clinician (e.g., a time sufficient to allow an overlap of the pharmaceutical effects of the therapies).

In some embodiments, a method described herein further includes administering to the subject a therapeutically effective amount of an additional therapy (e.g., an additional therapeutic agent, such as cisplatin).

### Summary

Etoposide is a lipophilic, low solubility, high impact therapeutic that could benefit from modification by glycosylation.

There is a need for DNA topoisomerase II inhibitors with improved aqueous solubility and with different PK/PD profiles to provide potential improvements in potency towards inhibiting the activity of the DNA topoisomerase II protein and enhanced anti-tumor effects.

### EXEMPLIFICATION

### Example 1

### Establishment of a Glycosyltransferase (GT) Library and Cell Lysate-Based Assay to Identify Drug-Modifying Glycosyltransferases

Although GTs are one of the largest enzyme families in nature, the natural substrate(s) of the majority of GTs is unknown. Therefore, to identify GTs that can use a non-native substrate such as etoposide is a nontrivial effort. A screening strategy was designed to address this need. The phylogenetic method was utilized to select a set of enzymes representing the structural and functional biodiversity of a desired functional GT class, uridine diphosphate (UDP) glycosyltransferases (UGTs), across different kingdoms and species. Based on the bioinformatics analysis, 328 UGTs were selected, including enzymes from different species of bacteria, fungus, plants, and human. To establish the GT library, the cDNA of the selected UGTs were produced by either nucleotide synthesis or by RT-PCR from the RNA of tissues expressing the UGTs. Each of the resulting UGT gene cDNA was cloned into the yeast TEF-promoter expression plasmid p426-TEF. The plasmids were individually transformed into wild-type yeast (Saccharomyces cerevisiae) strain BY4743. After auxotrophic selection, the yeast colonies expressing the recombinant UGT proteins were cultured, harvested, and lysed by CelLytic Y cell lysis reagent (Sigma-Aldrich). A cell-free cell lysate-based glycosylation assay was designed to screen for UGTs that are able to glycosylate the target substrate (see below for details). All UGTs were assayed in parallel on 96-well plates to allow for high throughput screening. The drug-modifying UGTs can be identified by the appearance of new peaks in HPLC analysis. The characteristics of the novel drug glycosides can be evaluated further by specialized assays.

### Example 2

### Synthesis of Etoposide-3″-O-D-Glucoside and Etoposide-4′-O-D-Glucoside Using the Cell Lysate-Based Assay

A GT library made according to Example 1 was screened to identify enzymes able to catalyze regiospecific glycosylation of etoposide when UDP-glucose was used as the sugar donor. Etoposide (final concentration=50 μM) was added to each well of a 96-well microtiter plate containing a unique UGT enzyme in the reaction mixture (50 mM Tris, pH 8.0, 10 mM UDP-glucose, and 20 μL recombinant UGT-expressing yeast cell lysate), and the reaction (total volume 100 μL) was allowed to proceed for 5 hours at 30° C., followed by termination of the modification reaction by quenching with 100 μL methanol. As a negative control, a reaction with the lysate of yeast harboring p426-TEF empty vector was carried out. The presence of the desired glycosylated product was determined by subjecting the contents of each well to HPLC analysis.

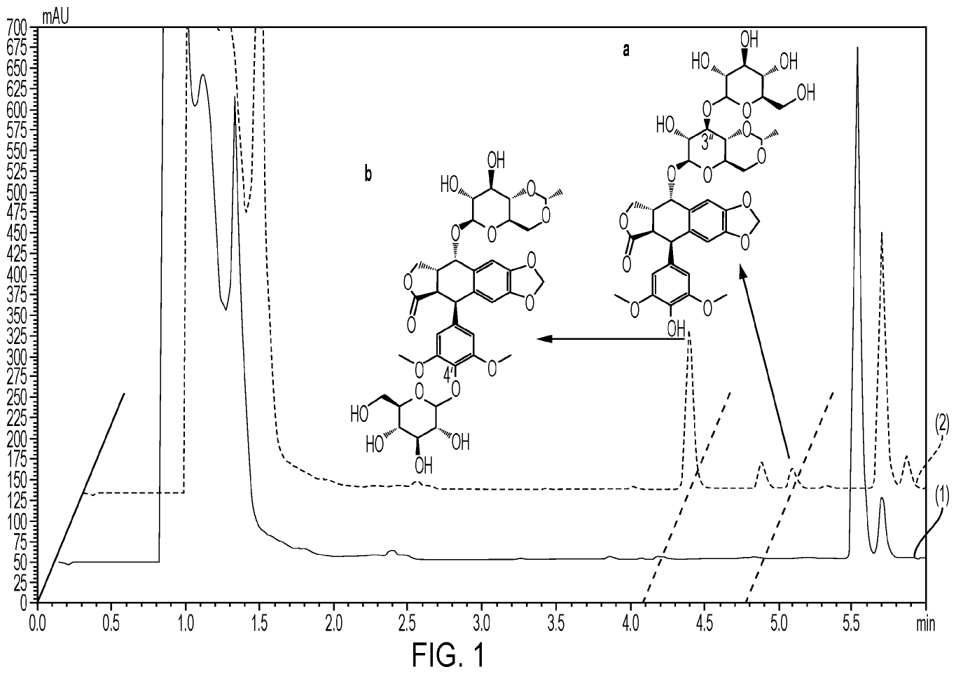

From the screen, three UGTs were able to modify etoposide when UDP-glucose was used as the sugar donor. The overall conversion rates are: 94% for SEQ ID NO: 1, 45% for SEQ ID NO: 2, 16% for SEQ ID NO: 5, 10% for SEQ ID NO: 3, 5% for SEQ ID NO: 4. Among the five UGTs, SEQ ID NO: 2 can produce both the monosaccharide etoposide-3″-O-D-glucoside (FIG. 1 chromatogram peak a) and the etoposide-4′-O-D-glucoside (FIG. 1 chromatogram peak b). SEQ ID NO: 3 can produce etoposide-3″-O-D-glucoside only. SEQ ID NO: 1, SEQ ID NO: 4 and SEQ ID NO: 5 can produce etoposide-4′-O-D-glucoside only.

The chemical identity of the etoposide glycosides was confirmed by LC-MS analysis: For a: m/z=749.41 [M−H]⁻, m/z=768.31 [M+NH₄]⁺, m/z=773.26 [M+Na]⁺; For b: m/z=795.31 [M+FA−H]⁻, m/z=768.25 [M+NH₄]⁺.

The chemical identity of the etoposide glycosides was further confirmed by nuclear magnetic resonance (NMR) analyses: For a (produced by SEQ ID NO: 2): ¹H NMR (DMSO-d₆, 600 MHz), δ 7.00 (1H, s), 6.53 (1H, s), 6.18 (2H, s), 6.02 (2H, d, J=13.8 Hz), 5.47 (1H, d, J=4.2 Hz), 4.94 (1H, d, J=3.0 Hz), 4.73 (1H, dd, J=10.2, 4.8 Hz), 4.65 (1H, d, J=7.8 Hz), 4.53 (1H, d, J=7.8 Hz), 4.48 (1H, d, J=5.4 Hz), 4.26 (1H, m), 4.09 (1H, dd, J=10.8, 4.8 Hz), 3.73 (1H, t, J=8.4 Hz), 3.71 (1H, d, J=4.8 Hz), 3.61 (6H, s), 3.52 (1H, t, J=10.2 Hz), 3.46 (1H, m), 3.36 (1H, t, J=9.0 Hz), 3.13 (1H, m), 3.07 (1H, m), 3.03 (1H, m), 2.98 (1H, m), 2.88(1H, m), 1.23 (3H, d, J=4.8 Hz). ¹³C NMR (DMSO-d₆, 150 MHz), δ 175.1, 148.2, 147.6, 146.6, 135.2, 133.3, 130.7, 129.1, 110.4, 110.3, 108.9, 103.3, 101.8, 101.3, 99.1, 80.6, 79.0, 77.4, 77.0, 74.6, 74.4, 72.5, 70.5, 68.1, 67.8, 66.0, 61.5, 56.5, 43.4, 40.9, 37.7, 20.7; For b (produced by SEQ ID NO: 1): ¹H NMR (DMSO-d₆, 500 MHz), δ 7.01 (1H, s), 6.54 (1H, s), 6.22 (2H, s), 6.02 (2H, d, J=3.0 Hz), 5.22 (1H, s), 5.21 (1H, s), 4.93 (2H, m), 4.88 (1H, d, J=5.0 Hz), 4.84 (1H, d, J=7.0 Hz), 4.81 (1H, d, J=3.5 Hz), 4.71 (1H, dd, J=10.0, 5.0 Hz), 4.57 (1H, d, J=8.0 Hz), 4.53 (1H, d, J=5.5 Hz), 4.26 (3H, m), 4.07 (1H, dd, J=10.5, 5.0 Hz), 3.57 (1H, m), 3.50 (1H, m), 3.40 (1H, m), 3.34 (1H, m), 3.24 (1H, m), 3.15 (4H, m), 3.04 (2H, m), 2.87 (1H, m), 1.23 (3H, d, J=5.0 Hz). ¹³C NMR (DMSO-d₆, 125 MHz), δ 175.1, 152.1, 148.2, 146.7, 136.2, 134.2, 132.8, 129.4, 110.4, 110.3, 109.7, 103.2, 102.0, 101.8, 99.1, 80.6, 77.5, 76.9, 74.9, 74.6, 73.2, 72.3, 70.3, 68.2, 67.8, 66.2, 61.3, 56.9, 43.4, 40.8, 37.7, 20.8.

The sequence of the enzymes identified as SEQ ID NOs.: 1-5 are disclosed herein in the sequences section.

### Example 3

### Synthesis of Etoposide-3″-O-D-Glucoside and Etoposide-4′-O-D-Glucoside Using Purified Recombinant Glycosyltransferases

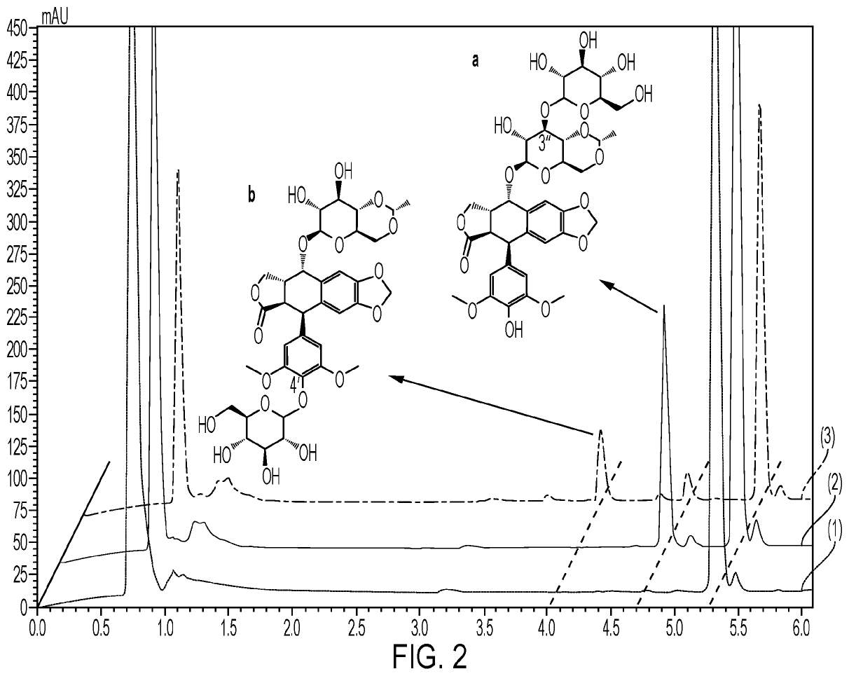

While a yeast cell lysate-based glycosylation assay is instrumental in initial screening efforts, one approach to producing larger amounts of ivacaftor glucosides is to use finely controlled enzyme concentrations during synthesis. To that end, two UGT genes identified in Example 2 (SEQ ID NO: 2 and 3) containing a metal-affinity purification tag at the C-terminus were transformed into BL21(DE3) Escherichia coli cells. Cells were grown at 37° C. until the cultures reached an optical density (OD600) of 0.5-0.8. Then, protein over-expression was induced with 1 mM isopropyl β-D-1-thiogalactopyranoside (IPTG) at 18° C. The culture was grown overnight for 16 hours and then harvested. Desired proteins were purified from the harvested cells using either free nickel-IDA resin or magnetic nickel-charged agarose beads. Ivacaftor glucoside synthesis using the purified recombinant enzymes was performed at volumes ranging from 10-75 mL. Etoposide (final concentration 0.5 mg/ml) was added to the reaction mixture (final concentrations of 50 mM HEPES, 50 mM KCl, pH 7.5, 2 mM UDP-glucose, 1 uM UGT), and the reaction was allowed to proceed for 1-3 days at 37° C. The reaction was terminated by adding 1 reaction volume of ice-cold methanol. The reaction was then incubated at 90° C. to ensure that the enzyme was adequately denatured. The presence of the desired glycosylated product(s) was determined by HPLC analysis (FIG. 2). From these reactions, SEQ ID NO: 2 and 3 can produce the monosaccharide etoposide-3″-O-D-glucoside (FIG. 2 chromatogram peak a). SEQ ID NO: 3 can also produce the monosaccharide etoposide-4′-O-D-glucoside (FIG. 2 chromatogram peak b).

The chemical identity of the etoposide glycosides was confirmed by LC-MS analysis: For a: m/z=751.12 [M+H]⁺, m/z=768.15 [M+NH₄]⁺; For b: m/z=751.15 [M+H]⁺, m/z=768.12 [M+NH₄]⁺.

The chemical identity of the etoposide-3″-O-D-glucoside produced using SEQ ID NO: 3 (FIG. 2 chromatogram peak a) was further confirmed by nuclear magnetic resonance (NMR) analyses, and the structure was determined to be the same as the etoposide-3″-O-D-glucoside produced using the cell lysate-based assay in Example 2 (FIG. 1 chromatogram peak a).

### Example 4

### Synthesis of Etoposide-3″-O-D-Galactoside and Etoposide-4′-O-D-Galactoside Using the Cell Lysate-Based Assay

A GT library made according to Example 1 was screened to identify enzymes able to catalyze regiospecific glycosylation of etoposide when UDP-galactose was used as the sugar donor. Etoposide (final concentration=50 μM) was added to each well of a 96-well microtiter plate containing a unique UGT enzyme in the reaction mixture (50 mM Tris, pH 8.0, 2 mM UDP-galactose and 20 μL recombinant UGT-expressing yeast cell lysate), and the reaction (total volume 100 μL) was allowed to proceed for 5 hours at 30° C., followed by termination of the modification reaction by quenching with 100 ∞L methanol. As a negative control, a reaction with the lysate of yeast harboring p426-TEF empty vector was carried out. The presence of the desired glycosylated product was determined by subjecting the contents of each well to HPLC analysis.

From the screen, three UGTs were able to modify etoposide when UDP-galactose was used as the sugar donor. The overall conversion rates are: 32% for SEQ ID NO: 1, 3% for SEQ ID NO: 2 and 2% for SEQ ID NO: 3. SEQ ID NO: 3 can produce monosaccharide etoposide-3″-O-D-galactoside. SEQ ID NO: 2 and SEQ ID NO: 1 can produce monosaccharide etoposide-4′-O-D-galactoside.

The chemical identity of the etoposide glycosides was confirmed by LC-MS analysis: m/z=768.30 [M+NH₄]⁺.

### Example 5

### Synthesis of Etoposide-O-D-Xyloside Using the Cell Lysate-Based Assay

A GT library made according to Example 1 was screened to identify enzymes able to catalyze regiospecific glycosylation of etoposide when UDP-xylose was used as the sugar donor. Etoposide (final concentration=50 μM) was added to each well of a 96-well microtiter plate containing a unique UGT enzyme in the reaction mixture (50 mM Tris, pH 8.0, 2 mM UDP-xylose and 20 μL recombinant UGT-expressing yeast cell lysate), and the reaction (total volume 100 μL) was allowed to proceed for 5 hours at 30° C., followed by termination of the modification reaction by quenching with 100 μL methanol. As a negative control, a reaction with the lysate of yeast harboring p426-TEF empty vector was carried out. The presence of the desired glycosylated product was determined by subjecting the contents of each well to HPLC analysis.

From the screen, two UGTs were able to modify etoposide when UDP-xylose was used as the sugar donor. The overall conversion rates are: 19% for SEQ ID NO: 1 and 5% for SEQ ID NO: 2. Both SEQ ID NO: 1 and SEQ ID NO: 3 can produce monosaccharide etoposide-O-D-xyloside.

The chemical identity of the etoposide glycosides was confirmed by LC-MS analysis: m/z=738.27 [M+NH₄]⁺.

### Example 6

### Synthesis of Etoposide-O-N-Acetylglucosamide Using Purified Recombinant Glycosyltransferases

The purified recombinant assay described in Example 3 was conducted with the following modification. UDP-N-acetylglucosamine was used instead of UDP-glucose resulting in a final reaction mixture containing 50 mM HEPES, 50 mM KCl, pH 7.5, 2 mM UDP-N-acetylglucosamine, 1 uM UGT, and 0.5 mg/ml etoposide. The presence of the desired glycosylated product(s) was determined by HPLC analysis.

From this assay, one UGT was able to modify etoposide when UDP-N-acetylglucosamine was used as the sugar donor. The overall conversion rate is: 4% for SEQ ID NO: 3. SEQ ID NO: 3 can produce monosaccharide etoposide-O-N-acetylglucosamide.

The chemical identity of the etoposide glycoside was confirmed by LC-MS analysis: m/z=792.14 [M+H]⁺, m/z=809.37 [M+NH₄]⁺.

### Example 7

### Comparison of the Water Solubility of Etoposide and Etoposide-3″-O-D-Glucoside

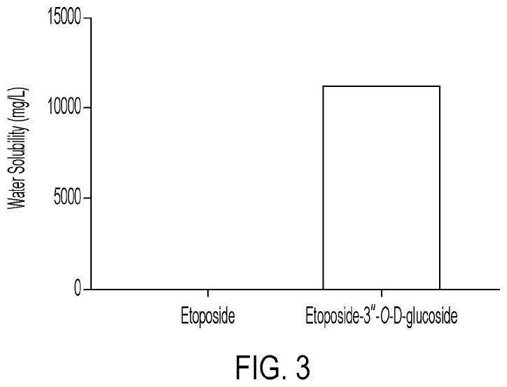

The water solubility of etoposide and etoposide-O-D-glucoside was investigated by suspending excess amounts of the two compounds in 200 μl of distilled water in a microcentrifuge tube at 25° C. for 12 h. Afterwards, each sample was centrifuged at 12,000×g for 20 min. The supernatant of each sample was then filtered through a 0.45-μm membrane filter and the concentration of the compound in the supernatant, which is defined as the water-soluble component, was measured by its absorbance at 254 nm using HPLC, and its absolute solubility was calculated in reference to the concentration-absorbance standard curve. As shown in FIG. 3, the water solubility of etoposide was determined to be 56 mg/L, whereas that of etoposide-3″-O-D-glucoside was 11200 mg/L, which is 200 times higher.

### REFERENCES

Abel, Mark, Roman Szweda, Daniel Trepanier, Randall W. Yatscoff, and Robert T. Foster. 2007. Rapamycin carbohydrate derivatives. U.S. Pat. No. 7,160,867 B2. US Patent, filed May 13, 2004, and issued Jan. 9, 2007.

American Cancer Society. Global Cancer Facts & FIGS. 4th Edition. Atlanta: American Cancer Society; 2018.

Arnold, A. M. 1979. “Podophyllotoxin Derivative VP 16-213.” Cancer Chemotherapy and Pharmacology 3 (2). https://doi.org/10.1007/bf00254976.

Arts, Ilja C. W., Aloys L. A. Sesink, Maria Faassen-Peters, and Peter C. H. Hollman. 2004. “The Type of Sugar Moiety Is a Major Determinant of the Small Intestinal Uptake and Subsequent Biliary Excretion of Dietary Quercetin Glycosides.” The British Journal of Nutrition 91 (6): 841-47.

Beijnen, J. H., J. J. M. Holthuis, H. G. Kerkdijk, O. A. G. J. van der Houwen, A. C. A. Paalman, A. Bult, and W. J. M. Underberg. 1988. “Degradation Kinetics of Etoposide in Aqueous Solution.” International Journal of Pharmaceutics 41 (1-2): 169-78.

Berman, H. M. 2000. “The Protein Data Bank.” Nucleic Acids Research 28 (1): 235-42.

Bonina, Francesco, Carmelo Puglia, Maria Grazia Rimoli, Daniela Melisi, Giampiero Boatto, Maria Nieddu, Antonio Calignano, Giovanna La Rana, and Paolo De Caprariis. 2003. “Glycosyl Derivatives of Dopamine and L-Dopa as Anti-Parkinson Prodrugs: Synthesis, Pharmacological Activity and in Vitro Stability Studies.” Journal of Drug Targeting 11 (1): 25-36.

Breton, Christelle, Sylvie Fournel-Gigleux, and Monica M. Palcic. 2012. “Recent Structures, Evolution and Mechanisms of Glycosyltransferases.” Current Opinion in Structural Biology 22 (5): 540-49.

De Bruyn, Frederik, Jo Maertens, Joeri Beauprez, Wim Soetaert, and Marjan De Mey. 2015. “Biotechnological Advances in UDP-Sugar Based Glycosylation of Small Molecules.” Biotechnology Advances 33 (2): 288-302.

“Etoposide.” (2005) 2020. DrugBank. Jan. 28, 2020. https://www.drugbank.ca/drugs/DB00773.

Fernandez, C., O. Nieto, E. Rivas, G. Montenegro, J. A. Fontenla, and A. Fernandez-Mayoralas. 2000. “Synthesis and Biological Studies of Glycosyl Dopamine Derivatives as Potential Antiparkinsonian Agents.” Carbohydrate Research 327 (4): 353-65.

Gantt, Richard W., Pauline Peltier-Pain, and Jon S. Thorson. 2011. “Enzymatic Methods for Glyco(diversification/randomization) of Drugs and Small Molecules.” Natural Product Reports 28 (11): 1811-53.

Gu, Xiangying, Lin Chen, Xin Wang, Xiao Liu, Qidong You, Wenwei Xi, Li Gao, et al. 2014. “Direct Glycosylation of Bioactive Small Molecules with Glycosyl Iodide and Strained Olefin as Acid Scavenger.” The Journal of Organic Chemistry 79 (3): 1100-1110.

Hande, K. R. 1998. “Etoposide: Four Decades of Development of a Topoisomerase II Inhibitor.” European Journal of Cancer 34 (10): 1514-21.

Hardman, Janee' M., Robert T. Brooke, and Brandon J. Zipp. 2017. “Cannabinoid Glycosides: In Vitro Production of a New Class of Cannabinoids with Improved Physicochemical Properties.” bioRxiv. https://doi.org/10.1101/104349.

Hatfield, Jason M., Monika Wierdl, Randy M. Wadkins, and Philip M. Potter. 2008. “Modifications of Human Carboxylesterase for Improved Prodrug Activation.” Expert Opinion on Drug Metabolism & Toxicology 4 (9): 1153-65.

Keilholz, U., L. Rohde, P. Mehlitz, M. Knoedler, A. Schmittel, V. Kümmerlen, K. Klinghammer, et al. 2017. “First-in-Man Dose Escalation and Pharmacokinetic Study of CAP7.1, a Novel Prodrug of Etoposide, in Adults with Refractory Solid Tumours.” European Journal of Cancer 80 (July): 14-25.

Kolar, Cenek, Jörg Czech, Klaus Bosslet, Gerhard Seemann, Hans-Harald Sedlacek, and Marburg Dieter Hoffman. 2004. Glycosyl-etoposide prodrugs, a Process for the Preparation Thereof and the Use Thereof in Combination with Functionalized Tumor-Specific Enzyme Conjugates. U.S. Pat. No. 7,241,595 B2. US Patent, filed Apr. 24, 2002, and issued Apr. 3, 2004.

Krebber S., Claus, Christopher Davis, Stephen Delcardayre, Sergey A. Selifonov, and Russell Howard. 2001. Evolution and use of enzymes for combinatorial and medicinal chemistry. WIPO WO/2001/012817 A1. World Patent, filed Aug. 11, 2000, and published Feb. 22, 2001.

Křen, Vladimír. 2008. “Glycoside vs. Aglycon: The Role of Glycosidic Residue in Biological Activity.” Glycoscience, 2589-2644.

Kren, V., and L. Martinková. 2001. “Glycosides in Medicine: ‘The Role of Glycosidic Residue in Biological Activity.’” Current Medicinal Chemistry 8 (11): 1303-28.

Lairson, L. L., B. Henrissat, G. J. Davies, and S. G. Withers. 2008. “Glycosyltransferases: Structures, Functions, and Mechanisms.” Annual Review of Biochemistry 77 (1): 521-55.

Lin, Yih-Shyan, Rudeewan Tungpradit, Supachok Sinchaikul, Feng-Ming An, Der-Zen Liu, Suree Phutrakul, and Shui-Tein Chen. 2008. “Targeting the Delivery of Glycan-Based Paclitaxel Prodrugs to Cancer Cells via Glucose Transporters.” Journal of Medicinal Chemistry 51 (23): 7428-41.

Liu, Der-Zen, Supachok Sinchaikul, Peddiahgari Vasu Govardhana Reddy, Meng-Yang Chang, and Shui-Tein Chen. 2007. “Synthesis of 2′-Paclitaxel Methyl 2-Glucopyranosyl Succinate for Specific Targeted Delivery to Cancer Cells.” Bioorganic & Medicinal Chemistry Letters 17 (3): 617-20.

Lombard, Vincent, Hemalatha Golaconda Ramulu, Elodie Drula, Pedro M. Coutinho, and Bernard Henrissat. 2014. “The Carbohydrate-Active Enzymes Database (CAZy) in 2013.” Nucleic Acids Research 42 (D1): D490-95.

Maanen, J. M. S. van, J. Retel, J. de Vries, and H. M. Pinedo. 1988. “Mechanism of Action of Antitumor Drug Etoposide: A Review.” JNCI Journal of the National Cancer Institute 80 (19): 1526-33.

Mikuni, Katsuhiko, Katsuyoshi Nakanishi, Koji Hara, Kozo Hara, Wakao Iwatani, Tetsuya Amano, Kosho Nakamura, Yoshinori Tsuchiya, Hiroshi Okumoto, and Tadakatsu Mandai. 2008. “In Vivo Antitumor Activity of Novel Water-Soluble Taxoids.” Biological & Pharmaceutical Bulletin 31 (6): 1155-58.

Mylan Pharmaceuticals Inc. 2016. “Etoposide (capsule) [package Insert].” NIH US National Library of Medicine. https://dailymed.nlm.nih.gov/dailymed/drugInfo.cfm?setid=508a418e-985f-4208-9324-2230655bb5c2.

“NCCN Chemotherapy Order Templates.” National Comprehensive Cancer Network. Accessed Mar. 9, 2020. https://www.nccn.org/professionals/OrderTemplates/Default.aspx.

Olthof, Margreet R., Peter C. H. Hollman, Tom B. Vree, and Martijn B. Katan. 2000. “Bioavailabilities of Quercetin-3-Glucoside and Quercetin-4′-Glucoside Do Not Differ in Humans.” The Journal of Nutrition 130 (5): 1200-1203.

Peltier-Pain, Pauline, Shannon C. Timmons, Agnes Grandemange, Etienne Benoit, and Jon S. Thorson. 2011. “Warfarin Glycosylation Invokes a Switch from Anticoagulant to Anticancer Activity.” Chem Med Chem 6 (8): 1347-50.

Polt, Robin, Muthu Dhanasekaran, and Charles M. Keyari. 2005. “Glycosylated Neuropeptides: A New Vista for Neuropsychopharmacology?” Medicinal Research Reviews 25 (5): 557-85.

Rezonja, Renata, Lea Knez, Tanja Cufer, and Aleš Mrhar. 2013. “Oral Treatment with Etoposide in Small Cell Lung Cancer—Dilemmas and Solutions.” Radiology and Oncology 47 (1): 1-13.

Schmid, Jochen, Dominik Heider, Norma J. Wendel, Nadine Sperl, and Volker Sieber. 2016. “Bacterial Glycosyltransferases: Challenges and Opportunities of a Highly Diverse Enzyme Class Toward Tailoring Natural Products.” Frontiers in Microbiology 7 (February): 182.

Shah, J. C., J. R. Chen, and D. Chow. 1989. “Preformulation Study of Etoposide: Identification of Physicochemical Characteristics Responsible for the Low and Erratic Oral Bioavailability of Etoposide.” Pharmaceutical Research 6 (5): 408-12.

Slevin, M. L., P. Clark, S. P. Joel, S. Malik, R. J. Osborne, W. M. Gregory, D. G. Lowe, R. H. Reznek, and P. F. Wrigley. 1990. “A Randomized Trial to Evaluate the Effect of Schedule on the Activity of Etoposide in Small-Cell Lung Cancer.” Lung Cancer 6 (1-2): 56-57.

Squibb, Bristol-Myers. 2019. “Etopophos (etoposide Phosphate) for Injection, for Intravenous Use [package Insert].” FDA . https://www.accessdata.fda.gov/drugsatfda_docs/label/2019/020457s0191b1.pdf.

Takechi, M., and Y. Tanaka. 1994. “Structure-Activity Relationships of Synthetic Digitoxigenyl Glycosides.” Phytochemistry 37 (5): 1421-23.

Terao, Junji, Sachiyo Yamaguchi, Mutsuko Shirai, Mariko Miyoshi, Jae-Hak Moon, Syunji Oshima, Takahiro Inakuma, Tojiro Tsushida, and Yoji Kato. 2001. “Protection by Quercetin and Quercetin 3-O-β-D-Glucuronide of Peroxynitrite-Induced Antioxidant Consumption in Human Plasma Low-Density Lipoprotein.” Free Radical Research 35 (6): 925-31.

Tian, Lingling, Haibing He, and Xing Tang. 2007. “Stability and Degradation Kinetics of Etoposide-Loaded Parenteral Lipid Emulsion.” Journal of Pharmaceutical Sciences 96 (7): 1719-28.

Toffoli, G., G. Corona, R. Sorio, I. Robieux, B. Basso, A. M. Colussi, and M. Boiocchi. 2001. “Population Pharmacokinetics and Pharmacodynamics of Oral Etoposide.” British Journal of Clinical Pharmacology 52 (5): 511-19.

Torrens-Spence, Michael P., Tomáš Pluskal, Fu-Shuang Li, Valentina Carballo, and Jing-Ke Weng. 2018. “Complete Pathway Elucidation and Heterologous Reconstitution of Rhodiola Salidroside Biosynthesis.” Molecular Plant 11 (1): 205-17.

Wrasidlo, Wolf, Ulrike Schröder, Kathrin Bernt, Nicole Hübener, Doron Shabat, Gerhard Gaedicke, and Holger Lode. 2002. “Synthesis, Hydrolytic Activation and Cytotoxicity of Etoposide Prodrugs.” Bioorganic & Medicinal Chemistry Letters 12 (4): 557-60.

Xiao, Jianbo, Hui Cao, Yuanfeng Wang, Jinyao Zhao, and Xinlin Wei. 2009. “Glycosylation of Dietary Flavonoids Decreases the Affinities for Plasma Protein.” Journal of Agricultural and Food Chemistry 57 (15): 6642-48.

Xie, Kebo, Ridao Chen, Dawei Chen, Jianhua Li, Ruishan Wang, Lin Yang, and Jungui Dai. 2017. “EnzymaticN-Glycosylation of Diverse Arylamine Aglycones by a Promiscuous Glycosyltransferase fromCarthamus Tinctorius.” Advanced Synthesis & Catalysis 359 (4): 603-8.

Yonekura-Sakakibara, Keiko, and Kousuke Hanada. 2011. “An Evolutionary View of Functional Diversity in Family 1 Glycosyltransferases.” The Plant Journal: For Cell and Molecular Biology 66 (1): 182-93.

Zhang, Yujiao, Kebo Xie, Aijing Liu, Ridao Chen, Dawei Chen, Lin Yang, and Jungui Dai. 2019. “Enzymatic Biosynthesis of Benzylisoquinoline Alkaloid Glycosides via Promiscuous Glycosyltransferases from Carthamus Tinctorius.” Chinese Chemical Letters 30 (2): 443-46.

Zhu, Xiangming, and Richard R. Schmidt. 2009. “New Principles for Glycoside-Bond Formation.” Angewandte Chemie 48 (11): 1900-1934.

Zipp, Brandon Joel, Janee M. Hardman, and Robert T. Brooke. 2018. Cannabinoid glycoside prodrugs and methods of synthesis. USPTO US 2018/0264122 A1. US Patent, published Sep. 20, 2018.

Altschul, S., Gish, W., Miller, W., Myers, E., and Lipman, D. (1990). Basic local alignment search tool. Journal of Molecular Biology. 215 (3): 403-4.

Henikoff, S. and J. G. Henikoff (1992). “Amino acid substitution matrices from protein blocks.” Proceedings of the National Academy of Sciences of the United States of America 89(22): 10915-10919.

### INCORPORATION BY REFERENCE; EQUIVALENTS

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

1. A compound represented by the following structural formula: 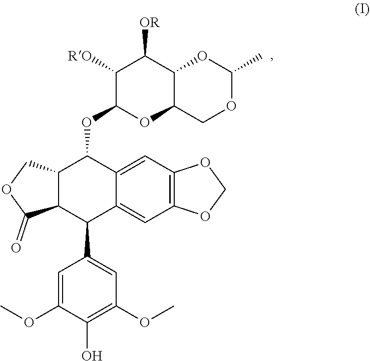
or a pharmaceutically acceptable salt thereof,
wherein R is a hydrogen, a monosaccharide, a disaccharide, a trisaccharide, or an oligosaccharide comprising 4 to 10 monosaccharides,
wherein R′ is a hydrogen, a monosaccharide, a disaccharide, a trisaccharide, or an oligosaccharide comprising 4 to 10 monosaccharides, and
wherein at least one of R and R′ is not hydrogen.

2. The compound of claim 1, wherein R is a monosaccharide.

3. The compound of claim 1, wherein R′ is a monosaccharide.

4. The compound of claim 1, wherein the monosaccharide is a pentose monosaccharide, hexose monosaccharide, or heptose monosaccharide.

5. The compound of claim 1, wherein R is allose, apiose, arabinose, fructose, fucitol, fucose, galactose, glucose, glucuronic acid, mannose, N-acetylglucosamine, N-acetylgalactosamine, rhamnose, or xylose.

6. The compound of claim 1, wherein R′ is allose, apiose, arabinose, fructose, fucitol, fucose, galactose, glucose, glucuronic acid, mannose, N-acetylglucosamine, N-acetylgalactosamine, rhamnose, or xylose.

7. The compound of claim 1, wherein R is glucosamine, galactosamine, mannosamine, 5-thio-D-glucose, nojirimycin, deoxynojirimycin, 1,5-anhydro-D-sorbitol, 2,5-anhydro-D-mannitol, 2-deoxy-D-galactose, 2-deoxy-D-glucose, 3-deoxy-D-glucose, arabinitol, galactitol, glucitol, iditol, lyxose, mannitol, L-rhamnitol, 2-deoxy-D-ribose, ribose, ribitol, ribulose, xylulose, altrose, gulose, idose, levulose, psicose, sorbose, tagatose, talose, galactal, glucal, fucal, rhamnal, arabinal, xylal, 3,4-di-O-acetyl-L-fucal, 3,4-di-O-acetyl-L-rhamnal, 3,4-di-O-acetyl-D-arabinal, 3,4-di-O-acetyl-D-xylal, valienamine, validamine, valiolamine, valienol, valienone, galacturonic acid, mannuronic acid, N-acetylneuraminic acid, N-acetylmuramic acid, gluconic acid D-lactone, galactonic acid gamma-lactone, galactonic acid delta-lactone, mannonic acid gamma-lactone, D-altro-heptulose, D-manno-heptulose, D-glycero-D-manno-heptose, D-glycero-D-gluco-heptose, D-allo-heptulose, D-altro-3-heptulose, D-glycero-D-manno-heptitol, or D-glycero-D-altro-heptitol.

8. The compound of claim 1, wherein R′ is glucosamine, galactosamine, mannosamine, 5-thio-D-glucose, nojirimycin, deoxynojirimycin, 1,5-anhydro-D-sorbitol, 2,5-anhydro-D-mannitol, 2-deoxy-D-galactose, 2-deoxy-D-glucose, 3-deoxy-D-glucose, arabinitol, galactitol, glucitol, iditol, lyxose, mannitol, L-rhamnitol, 2-deoxy-D-ribose, ribose, ribitol, ribulose, xylulose, altrose, gulose, idose, levulose, psicose, sorbose, tagatose, talose, galactal, glucal, fucal, rhamnal, arabinal, xylal, 3,4-di-O-acetyl-L-fucal, 3,4-di-O-acetyl-L-rhamnal, 3,4-di-O-acetyl-D-arabinal, 3,4-di-O-acetyl-D-xylal, valienamine, validamine, valiolamine, valienol, valienone, galacturonic acid, mannuronic acid, N-acetylneuraminic acid, N-acetylmuramic acid, gluconic acid D-lactone, galactonic acid gamma-lactone, galactonic acid delta-lactone, mannonic acid gamma-lactone, D-altro-heptulose, D-manno-heptulose, D-glycero-D-manno-heptose, D-glycero-D-gluco-heptose, D-allo-heptulose, D-altro-3-heptulose, D-glycero-D-manno-heptitol, or D-glycero-D-altro-heptitol.

9. The compound of claim 1, wherein R is a disaccharide.

10. The compound of claim 1, wherein R′ is a disaccharide.

11. The compound of claim 9, wherein R is a disaccharide of two glucose molecules.

12. The compound of claim 9, wherein R′ is a disaccharide of two glucose molecules.

13. The compound of claim 9, wherein R is a disaccharide of two galactose molecules.

14. The compound of claim 9, wherein R′ is a disaccharide of two galactose molecules.

15. The compound of claim 9, wherein R is a disaccharide of two xylose molecules.

16. The compound of claim 9, wherein R′ is a disaccharide of two xylose molecules.

17. The compound of claim 9, wherein the disaccharide molecules are bonded by a 1→2 glycosidic bond.

18. The compound of claim 9, wherein the disaccharide molecules are bonded by a 1→3 glycosidic bond.

19. The compound of claim 9, wherein the disaccharide molecules are bonded by a 1→4 glycosidic bond.

20. The compound of claim 1, wherein R or R′ is a trisaccharide.

21. The compound of claim 20, wherein R or R′ is a trisaccharide of three glucose molecules.

22. The compound of claim 20, wherein R or R′ is a trisaccharide of three galactose molecules.

23. The compound of claim 20, wherein R or R′ is a trisaccharide of three xylose molecules.

24. The compound of claim 20, wherein the trisaccharide molecules are bonded by a 1→2 glycosidic bond and by a 1→4 glycosidic bond.

25. (canceled)

26. A method of making an etoposide glycoside, the method comprising:
a) providing a reaction mixture comprising:
i) a compound having the following structural formula: 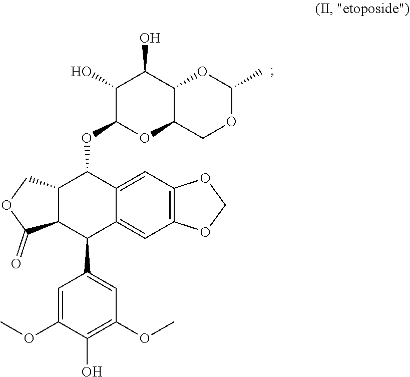
ii) a uridine diphosphate glycosyltransferase (UGT); and
iii) uridine diphosphate-monosaccharide;
b) allowing the reaction mixture to convert etoposide to a monosaccharide, a disaccharide, or an oligosaccharide of etoposide having the following structural formula: 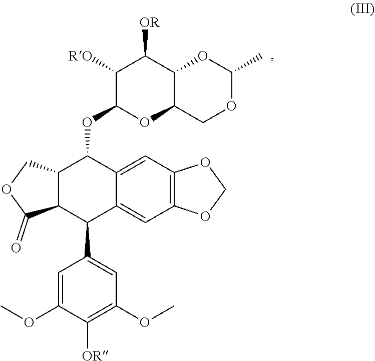
wherein R is a hydrogen, a monosaccharide, a disaccharide, a trisaccharide, or an oligosaccharide comprising 4 to 10 monosaccharides,
wherein R′ is a hydrogen, a monosaccharide, a disaccharide, a trisaccharide, or an oligosaccharide comprising 4 to 10 monosaccharides,
wherein R″ is a hydrogen, a monosaccharide, a disaccharide, a trisaccharide, or an oligosaccharide comprising 4 to 10 monosaccharides, and
wherein at least one of R, R′, and R″ is not hydrogen.

27. The method of claim 26, wherein the UGT comprises an amino acid sequence that is at least 95% similar to SEQ ID NO: 1.

28. The method of claim 26, wherein the UGT comprises an amino acid sequence that is at least 80% similar to a region from A340 to Q382 of SEQ ID NO: 1.

29. The method of claim 26, wherein the UGT comprises an amino acid sequence that is:
a) at least 90% similar to a region from 184 to S99 of SEQ ID NO: 1;
b) at least 90% similar to a region from D126 to F134 of SEQ ID NO: 1;
c) at least 90% similar to a region from L147 to S149 of SEQ ID NO: 1; and
d) at least 80% similar to a region from A340 to Q382 of SEQ ID NO: 1.

30. The method of claim 26, wherein the UGT comprises an amino acid sequence that is at least 95% similar to SEQ ID NO: 2.

31. The method of claim 26, wherein the UGT comprises an amino acid sequence that is at least 80% similar to a region from V278 to Q318 of SEQ ID NO: 2.

32. The method of claim 26, wherein the UGT comprises an amino acid sequence that is:
a) at least 90% similar to a region from I67 to D75 of SEQ ID NO: 2;
b) at least 90% similar to a region from D106 to L114 of SEQ ID NO: 2;
c) at least 90% similar to a region from C127 to S129 of SEQ ID NO: 2; and
d) at least 80% similar to a region from V278 to Q318 of SEQ ID NO: 2.

33. The method of claim 26, wherein the UGT comprises an amino acid sequence that is at least 95% similar to SEQ ID NO: 3.

34. The method of claim 26, wherein the UGT comprises an amino acid sequence that is at least 80% similar to a region from V291 to Q331 of SEQ ID NO: 3.

35. The method of claim 26, wherein the UGT comprises an amino acid sequence that is:
a) at least 90% similar to a region from W74 to V82 of SEQ ID NO: 3;
b) at least 90% similar to a region from D111 to V119 of SEQ ID NO: 3;
c) at least 90% similar to a region from F132 to N134 of SEQ ID NO: 3; and
d) at least 80% similar to a region from V291 to Q331 of SEQ ID NO: 3.

36. The method of claim 26, wherein the UGT comprises an amino acid sequence that is at least 95% similar to SEQ ID NO: 4.

37. The method of claim 26, wherein the UGT comprises an amino acid sequence that is at least 80% identical to a region from V280 to Q320 of SEQ ID NO: 4.

38. The method of claim 26, wherein the UGT comprises an amino acid sequence that is:
a) at least 90% similar to a region from I67 to D75 of SEQ ID NO: 4;
b) at least 90% similar to a region from D106 to L114 of SEQ ID NO: 4;
c) at least 90% similar to a region from C127 to S129 of SEQ ID NO: 4; and
d) at least 80% similar to a region from V280 to Q320 of SEQ ID NO: 4.

39. The method of claim 26, wherein the UGT comprises an amino acid sequence that is at least 95% similar to SEQ ID NO: 5.

40. The method of claim 26, wherein the UGT comprises an amino acid sequence that is at least 80% identical to a region from V283 to Q323 of SEQ ID NO: 5.

41. The method of claim 26, wherein the UGT comprises an amino acid sequence that is:
a) at least 90% similar to a region from I67 to Q79 of SEQ ID NO: 5;
b) at least 90% similar to a region from D110 to L118 of SEQ ID NO: 5;
c) at least 90% similar to a region from C131 to T133 of SEQ ID NO: 5; and
d) at least 80% similar to a region from V283 to Q323 of SEQ ID NO: 5.

42. The method of claim 26, wherein the uridine diphosphate-monosaccharide is uridine diphosphate-glucose (“UDP-glucose”).

43. The method of claim 26, wherein the uridine diphosphate-monosaccharide is uridine diphosphate-galactose (“UDP-galactose”).

44. The method of claim 26, wherein the uridine diphosphate-monosaccharide is uridine diphosphate-xylose (“UDP-xylose”).

45. The method of claim 26, wherein the uridine diphosphate-monosaccharide is uridine diphosphate-N-acetylglucosamine (“UDP-N-acetylglucosamine”).

46. A method of treating cancer, the method comprising administering to a patient in need thereof a therapeutically effective amount of a compound having the following structural formula: 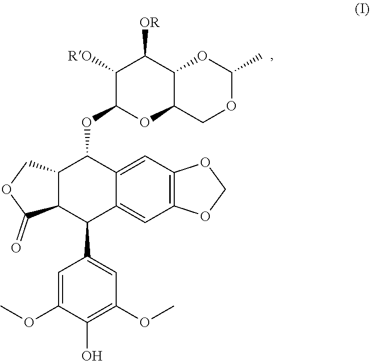
or a pharmaceutically acceptable salt thereof,
wherein R is a hydrogen, a monosaccharide, a disaccharide, a trisaccharide, or an oligosaccharide comprising 4 to 10 monosaccharides,
wherein R′ is a hydrogen, a monosaccharide, a disaccharide, a trisaccharide, or an oligosaccharide comprising 4 to 10 monosaccharides, and
wherein at least one of R and R′ is not hydrogen.

47.-50. (canceled)


SEQUENCE LISTING (5 sequences)

SEQ ID NO: 1 - protein, 465 aa, Galega orientalis
MAMNEMNKKAELIFIPAPGFGHLTPALEFAKLLTNLNNNLYITILCIKLPNTPNSDSYMKSVLASQPHIKLIDLPDVEEPPQELLNKSPEFFILTLMESLVPHVKKTIQTILSSTSSNKIVGLVLDFFCVSMIDVGNKLNIPSYLFLTSNVGFLSLMLSLQNRRIEDVFNDSNNHDQILIHGFSNSVPTNVLPDAVFNKDGGYIAYYKLAERFRDTKGIIVNTFSDLEQYSIDLLYDHDEKIPPIYAVGPLIDLKGSPNPNLDQSQHDLILKWLDEQPNKSVVFLCFGSFGFSFSPSQTREIALGLKHSGVRFLWTMKSPPEGLLEKMELEGKGMICGWAPQVEVLAHKAIGGFVSHCGWNSILESLWFGVPIFTWPIYAEQQLNAFKIVKEWGIALELRLNYRRGSDVVVAEEIEKGLKNLMDRDNVMHKKVQEMKDMARNAVVDGGSSFISVGKFIDDIIGNL

SEQ ID NO: 2 - protein, 392 aa, Bacillus subtilis
MKKYHISMINIPAYGHVNPTLALVEKLCEKGHRVTYATTEEFAPAVQQAGGEALIYHTSLNIDPKQIREMMEKNDAPLSLLKESLSILPQLEELYKDDQPDLIIYDFVALAGKLFAEKLNVPVIKLCSSYAQNESFQLGNEDMLKKIREAEAEFKAYLEQEKLPAVSFEQLAVPEALNIVFMPKSFQIQHETFDDRFCFVGPSLGERKEKESLLIDKDDRPLMLISLGTAFNAWPEFYKMCIKAFRDSSWQVIMSVGKTIDPESLEDIPANFTIRQSVPQLEVLEKADLFISHGGMNSTMEAMNAGVPLVVIPQMYEQELTANRVDELGLGVYLPKEEVTVSSLQEAVQAVSSDQELLSRVKNMQKDVKEAGGAERAAAEIEAFMKKSAVPQ

SEQ ID NO: 3 - protein, 415 aa, Streptomyces antibioticus
MTTQTTPAHIAMFSIAAHGHVNPSLEVIRELVARGHRVTYAIPPVFADKVAATGARPVLYHSTLPGTDADPEAWGSTLLDNVEPFLNDAIQALPQLADAYADDIPDLVLHDITSYPARVLARRWGVPAVSLFPNLVAWKGYEEEVAEPMWREPRQTERGRAYYARFEAWLKENGITEHPDTFASHPPRSLVLIPKALQPHADRVDEDVYTFVGACQGDRAEEGGWQRPAGAEKVVLVSLGSVFTKQPAFYRECVRAFGNLPGWHLVLQIGRKVTPAELGELPDNVEVHDWVPQLAILRQADLFVTHAGAGGSQEGLATATPMIAVPQAVDQFGNADMLQGLGVARKLATEEATADLLRETALALVDDPEVARRLRRIQAEMAQEGGTRRAADLIEAELPARHERQEPVGDRPNGG

SEQ ID NO: 4 - protein, 394 aa, Bacillus methylotrophicus
MRKTHIAIINVAAHGHVNPTLPVAEELVNRGYKVTFATTEEFEASVAKTGAIPVLYRTSIKADPETIKERVNKNDAFVMFLEEAVEVLPQLEELYKDDLPDIVLFDFLALAGRLFADNGGIPAVKFCPSYAMNEYFQLGRDEETLEAAKQAIEEYQEQIENEQLKRMTMEEFFMPEKLNIVFMPRAFQPKQETFDERFCFVGPSLGERTNTGSLEIDAADDRPLMLISLGTAFNAWPEFYRMCIDAFRDSDWRVIMSVGTTIDPESFSDVPDHFIIRQHVPQLDVLEKAKLFVSHGGMNSTMEAMNAGVPLVVVPQMHEQEVTAKRVDELGLGVHLPPKEVTVARLQKDVQNVYGDKNILSRVKSMQQKIEETGGPKQAVCAIEEFLKTAAVEN

SEQ ID NO: 5 - protein, 396 aa, Bacillus licheniformis
MGHKHIAIFNIPAHGHINPTLALTASLVKRGYRVTYPVTDEFVKAVEETGAEPLNYRSTLNIDPQQIRELMKNKKDMSQAPLMFIKEMEEVLPQLEALYENDKPDLILFDFMAMAGKLLAEKFGIEAVRLCSTYAQNEHFTFRSISEEFKIELTPEQEDALKNSNLPSFNFEDMFEPAKLNIVFMPRAFQPYGETFDERFSFVGPSLAKRKFQEKETPIISDSGRPVMLISLGTAFNAWPEFYHMCIEAFRDTKWQVIMAVGTTIDPESFDDIPENFSIHQRVPQLEILKKAELFITHGGMNSTMEGLNAGVPLVAVPQMPEQEITARRVEELGLGKHLQPEDTTAASLREAVSQTDGDPHVLKRIQDMQKHIKQAGGAEKAADEIEAFLAPAGVK